(19) United States
(12) Patent Application Publication
(10) Pub. No.: US 2026/0306284 A1
(43) Pub. Date: Oct. 8, 2026

(54) SYSTEMS, DEVICES, AND METHODS OF TREATING A NARROWING OF A BLOOD VESSEL

(71) Applicant: Route 92 Medical, Inc., San Mateo, CA (US)

(72) Inventors: Joey English, San Mateo, CA (US); Tony M. Chou, San Mateo, CA (US); John Miller, San Mateo, CA (US); Warren T. Kim, San Mateo, CA (US); Fabio Settecase, Mateo, CA (US); Matthew Alexander, San Mateo, CA (US); Michi E. Garrison, San Mateo, CA (US)

(21) Appl. No.: 19/709,934

(22) Filed: Jun. 16, 2026

Related U.S. Application Data
(63) Continuation-in-part of application No. 18/311,797, filed on May 3, 2023.
(60) Provisional application No. 63/338,114, filed on May 4, 2022.
(60) Provisional application No. 63/346,524, filed on May 27, 2022.

(51) Int. Cl. A61F 2/82; A61M 1/00; A61M 29/00

(52) U.S. Cl. CPC A61F 2/82; A61M 1/84; A61M 29/00; A61M 2202/04; A61M 2210/12

(57) ABSTRACT

Methods of treating a blood vessel that has a narrowed portion including navigating a catheter to the narrowed portion, a proximal section of the catheter having a non-expandable cylindrical surface having an outer diameter greater than an outer diameter of a tapered portion of a distal tip. Advancing the catheter within the narrowed portion so that the tapered portion of the distal tip passes at least partially through the narrowed portion to dilate the narrowed portion; and maintaining the catheter in a dilating position within the narrowed portion for a dwell time selected so that at least a portion of the narrowed portion is less narrow following removal of the catheter than the narrowed portion was prior to inserting the distal tip of the catheter into the narrowed portion. Related catheters, catheter systems, and methods are provided.

### CROSS REFERENCE TO RELATED APPLICATIONS

This application is a continuation-in-part of U.S. patent application Ser. No. 18/311,797, filed May 3, 2023, which claims the benefit of priority under 35 U.S.C. § 119(e) to U.S. Provisional Patent Application Ser. No. 63/346,524, filed May 27, 2022, and U.S. Provisional Patent Application Ser. No. 63/338,114, filed May 4, 2022. The disclosures are hereby incorporated by reference in their entireties.

### FIELD

The present technology relates to systems, devices, and methods of treating a narrowing of a blood vessel, more particularly, a narrowing due to atherosclerosis disease of blood vessels within the brain, leading to the brain, or coming from the brain.

### BACKGROUND

Various blood vessel conditions impair blood flow through the vessels of the brain. Acute ischemic stroke (AIS) is the sudden blockage of adequate blood flow to a section of the brain, usually caused by an occlusion, such as emboli lodging and/or thrombus forming in situ, in one of the blood vessels supplying the brain. Occlusions that are a result of atherosclerotic disease, such as intracranial atherosclerotic disease (ICAD) also impair blood flow within the brain.

The endovascular treatment in the vessels leading to the brain (e.g., cervical internal carotid arteries) and in the vessels of the brain (e.g., cerebral arteries) remains challenging. Acutely symptomatic intracranial atherosclerotic disease (ICAD) has been treated by balloon angioplasty to improve blood flow when medical management, including blood thinner medication, has failed. The vasculature of the skull is small and tortuous and there is a high risk of complications due to balloon angioplasty and thromboembolic complications due to the release of thrombotic material during treatments that can block blood flow to branch vessels. In addition, navigating through the delicate intracranial cerebral vessels that are highly stenotic can be risky because the delicate vessels can become inadvertently dissected, torn, or ruptured by the balloon catheter and the size and direction of the vessels are not well-visualized.

The time required to access sites of occlusions to restore, even partially, flow to the vessel is crucial in determining a successful outcome of treatment. Similarly, the occurrence of distal emboli during treatments and the potentially negative neurologic effect and procedural complications, such as perforation and intracerebral hemorrhage are limits to success of treatments.

There is a need for improved treatments of occlusions in vessels leading to the brain and vessels within the brain that improve outcomes with minimal risk of trauma.

### SUMMARY

In an aspect, disclosed is a method of treating a blood vessel that has been narrowed by atherosclerotic disease. The blood vessel is a carotid blood vessel or a cerebral blood vessel. The method includes navigating a catheter through a patient's vasculature to a narrowed portion of the blood vessel. The catheter has a proximal section and a distal tip having a tapered portion. The proximal section has a cylindrical surface having an outer diameter greater than an outer diameter of the tapered portion of the distal tip. The proximal section is capable of advancing the catheter through the patient's vasculature. The distal tip has a size and a flexibility to reach the narrowed portion of the blood vessel. The method includes inserting the distal tip of the catheter into the narrowed portion of the blood vessel; advancing the catheter within the narrowed portion so that the tapered portion of the distal tip passes at least partially through the narrowed portion to dilate the narrowed portion; and maintaining the catheter in a dilating position within the narrowed portion for a dwell time selected so that at least a portion of the narrowed portion is less narrow following removal of the catheter than the narrowed portion was prior to inserting the distal tip of the catheter into the narrowed portion.

The distal tip can be non-expandable. The dilating position can be a position where the cylindrical surface of the proximal section is advanced into at least a portion of the narrowed portion. The cylindrical surface can be non-expandable. The dilating position can be a position where the tapered portion is advanced entirely through the narrowed portion and the cylindrical surface of the proximal section is advanced into the narrowed portion. The outer diameter of the cylindrical surface can be selected in relation to a diameter of the blood vessel when not narrowed and/or in relation to a characteristic of the atherosclerotic disease. The dwell time can be greater than about 1 second. The dwell time can be from about 10 seconds to about 200 seconds. The dwell time can be from about 60 seconds to about 120 seconds.

The method can optionally include removing the tapered portion of the distal tip from at least a portion of the narrowed portion of the blood vessel, readvancing the catheter within the narrowed portion, and maintaining the catheter in the dilating position a second time. The method can include removing the tapered portion of the distal tip from the narrowed portion of the blood vessel, assessing the dilation of the narrowed portion, and readvancing the catheter within the narrowed portion if the dilation is not a desired amount, and maintaining the catheter in the dilating position a second time. The method can include removing the tapered portion of the distal tip from the narrowed portion of the blood vessel, assessing the dilation of the narrowed portion by injecting contrast medium, and readvancing the catheter within the narrowed portion if the dilation is not a desired amount, and maintaining the catheter in the dilating position a second time. The method can include removing the tapered portion of the distal tip from at least a portion of the narrowed portion of the blood vessel, and repeatedly advancing the catheter within the narrowed portion and maintaining the catheter in the dilating position until a desired dilation of the narrowed portion of the blood vessel upon removal of the distal tip from the narrowed portion is achieved.

Advancing the catheter within the narrowed portion can include advancing the catheter without a guidewire. Advancing the catheter within the narrowed portion can include advancing the catheter over a guidewire that extends through the narrowed portion, the guidewire being received within a lumen of the catheter. The lumen can have a diameter less than about 0.024″. The lumen can have a diameter of from about 0.014″ to about 0.024″.

The catheter can be a first catheter and the method further include removing the first catheter from the narrowed portion and navigating a second catheter through the patient's vasculature to the narrowed portion of the blood vessel. The second catheter can have a proximal section and a distal tip having a tapered portion. The proximal section of the second catheter can have a cylindrical surface having an outer diameter greater than the outer diameter of the cylindrical surface of the first catheter and greater than an outer diameter of the tapered portion of the distal tip of the second catheter. The proximal section can be capable of advancing the second catheter through the patient's vasculature. The distal tip of the second catheter can have a size and a flexibility to reach the narrowed portion of the blood vessel. The method can include inserting the distal tip of the second catheter into the narrowed portion of the blood vessel that has been dilated by the first catheter; advancing the second catheter within the narrowed portion so that the tapered portion of the distal tip of the second catheter passes at least partially through the narrowed portion to dilate the narrowed portion; and maintaining the second catheter in a dilating position within the narrowed portion for a dwell time selected so that at least a portion of the narrowed portion is less narrow following removal of the second catheter than it was prior to insertion of the second catheter into the narrowed portion.

The first catheter can be removed from the patient's vasculature before navigating a second catheter through the patient's vasculature. The outer diameter of the cylindrical surface of the first catheter can be from about 0.039 inches (1.0 mm) to about 0.071 inches (1.8 mm), and wherein the outer diameter of the cylindrical surface of the second catheter is from about 0.055 inches (1.4 mm) to about 0.088 inches (2.2 mm). The proximal section can include a tubular portion and a proximal extension extending proximally from the tubular portion. The proximal extension can be less flexible than the tubular portion. The distal tip can be from about 1 cm to about 5 cm long. The tapered portion of the distal tip can be from about 0.5 cm to about 4 cm long. The distal tip can include a first radiopaque marker positioned to indicate a distal end of the distal tip and a second radiopaque marker positioned to indicate a proximal end of the tapered portion. The distal tip can include a cylindrical extension extending distal to the tapered portion. The distal tip can be formed of a material having a material hardness of no greater than about Shore 35D. The tapered portion of the distal tip can taper from a first outer diameter to a second outer diameter that is smaller than the first outer diameter. The first outer diameter can be at least about 1.5 times the second outer diameter. The tapered portion of the distal tip can taper from a first outer diameter to a second outer diameter that is smaller than the first outer diameter. The first outer diameter can be from about 0.048 inches (1.22 mm) to about 0.080 inches (2.03 mm).

The catheter can be an inner catheter that is receivable within a lumen of an outer catheter. The outer catheter can be navigated to the narrowed portion of the blood vessel. The inner catheter can be advanced from the lumen of the outer catheter into the narrowed portion of the blood vessel. The outer catheter and the inner catheter can be navigated to the narrowed portion of the blood vessel together. The outer catheter can have a distal end and wherein the distal tip of the inner catheter extends distal to the distal end of the outer catheter and the proximal section of the inner catheter is within the lumen of the outer catheter during navigation. The inner catheter can be navigated to the narrowed portion of the blood vessel, and the outer catheter is then navigated to the narrowed portion over the inner catheter.

The narrowed portion can be a carotid artery stenosis.

The method can optionally include treating an occlusion in a cerebral blood vessel distal to the narrowed portion. The method can optionally include navigating the catheter through the narrowed portion to a site of a cerebral blood vessel occlusion and treating the cerebral blood vessel occlusion. Treating the cerebral blood vessel occlusion can optionally treating with an aspiration catheter navigated to the cerebral blood vessel occlusion over the catheter. The method can optionally include deploying a stent in the narrowed portion. The method can optionally include deploying a stent in the narrowed portion before or after treating the cerebral blood vessel occlusion. The method can optionally include deploying a stent in the narrowed portion after treating the cerebral blood vessel occlusion during the same surgical procedure.

The blood vessel can be a carotid artery or cerebral artery. The carotid artery can be a common carotid artery, an internal carotid artery, or an external carotid artery. The cerebral artery can be a middle cerebral artery, an anterior cerebral artery, or a posterior cerebral artery.

In an interrelated implementation, provided is a system for treating a carotid or cerebral blood vessel that has been narrowed. The system includes a first catheter having a proximal section and a distal tip having a tapered portion. The proximal section has a cylindrical surface having an outer diameter greater than an outer diameter of the tapered portion of the distal tip. The proximal section is capable of advancing the first catheter through a patient's vasculature to a narrowed portion of the carotid or cerebral blood vessel. The distal tip has a size and a flexibility to reach the narrowed portion. The system includes a second catheter having a proximal section and a distal tip having a tapered portion. The proximal section has a cylindrical surface having an outer diameter greater than an outer diameter of the tapered portion of the distal tip. The proximal section is capable of advancing the second catheter through the patient's vasculature to the narrowed portion of the carotid or cerebral blood vessel, and the distal tip has a size and a flexibility to reach the narrowed portion. The system includes an outer catheter having a lumen sized to receive the second catheter. The outer catheter is capable of being navigated to the narrowed portion of the blood vessel over the second catheter or together with the second catheter. The outer diameter of the cylindrical surface of the second catheter is greater than the outer diameter of the cylindrical surface of the first catheter so that the distal tip of the first catheter is insertable into the narrowed portion of the carotid or cerebral blood vessel, advanced to a dilating position, and removed from the narrowed portion and thereafter the second catheter is insertable into the narrowed portion of the carotid or cerebral blood vessel and advanced to a dilating position.

The outer catheter is optionally adapted to be advanced through the narrowed portion dilated by at least one of the first catheter and the second catheter to a cerebral occlusion distal to the narrowed portion to treat the cerebral occlusion. The outer catheter is optionally an aspiration catheter. The outer catheter is optionally adapted to deliver a stent to the narrowed portion. The outer catheter is optionally adapted to be advanced through the narrowed portion to a cerebral occlusion distal to the narrowed portion to treat the cerebral occlusion and to deliver a stent to the narrowed portion. The outer catheter and the second catheter are optionally adapted to be navigated together with the distal tip of the second catheter extending distal to a distal end of the outer catheter.

The distal tip of the first catheter can be non-expandable. The distal tip of the second catheter can be non-expandable. The cylindrical surface of the first catheter can be non-expandable. The cylindrical surface of the second catheter can be non-expandable. The outer diameter of the cylindrical surface of the first catheter can be from about 0.039 inches (1.0 mm) to about 0.071 inches (1.8 mm). The outer diameter of the cylindrical surface of the second catheter can be from about 0.055 inches (1.4 mm) to about 0.088 inches (2.2 mm).

The proximal section of the first catheter can include a tubular portion and a proximal extension extending proximally from the tubular portion, the proximal extension being less flexible than the tubular portion. The proximal section of the second catheter can include a tubular portion and a proximal extension extending proximally from the tubular portion of the second catheter, the proximal extension of the second catheter being less flexible than the tubular portion of the second catheter.

The distal tip of the first catheter can be from about 1 cm to about 5 cm long. The tapered portion of the distal tip of the first catheter can be from about 0.5 cm to about 4 cm long.

The distal tip of the first catheter can include a first radiopaque marker positioned to indicate a distal end of the distal tip of the first catheter and a second radiopaque marker positioned to indicate a proximal end of the tapered portion of the first catheter. The distal tip of the first catheter can include a cylindrical extension extending distal to the tapered portion of the first catheter.

The distal tip of the first catheter can be formed of a material having a material hardness of no greater than about Shore 35D. The tapered portion of the distal tip of the first catheter can taper from a first outer diameter to a second outer diameter that is smaller than the first outer diameter, the first outer diameter being at least about 1.5 times the second outer diameter. The tapered portion of the distal tip of the first catheter can taper from a first outer diameter to a second outer diameter that is smaller than the first outer diameter, the first outer diameter being from about 0.048 inches (1.22 mm) to about 0.080 inches (2.03 mm). The proximal section of the first catheter and the proximal section of the second catheter each can include a tubular portion and a proximal extension extending proximally from the tubular portion, the proximal extension being less flexible than the tubular portion. The distal tip of the first catheter and the distal tip of the second catheter can be from about 1 cm to about 5 cm long. The tapered portion of the distal tip of the first catheter and the distal tip of the second catheter can be from about 0.5 cm to about 4 cm long.

The distal tip of the first catheter can include a first radiopaque marker positioned to indicate a distal end of the distal tip of the first catheter and a second radiopaque marker positioned to indicate a proximal end of the tapered portion of the first catheter, the distal tip of the second catheter being a first marker band positioned to indicate a distal end of the distal tip of the second catheter and a second marker band positioned to indicate a proximal end of the tapered portion of the second catheter. The distal tip of the first catheter and/or the distal tip of the second catheter can optionally include a cylindrical extension extending distal to the tapered portion of the first catheter. The distal tip of the first catheter and the distal tip of the second catheter can be formed of a material having a material hardness of no greater than about Shore 35D. The tapered portion of the distal tip of the first catheter and the tapered portion of the distal tip of the second catheter can taper from a first outer diameter to a second outer diameter that is smaller than the first outer diameter, the first outer diameter being at least about 1.5 times the second outer diameter. The first catheter and the second catheter can be packaged together in a kit.

In an interrelated implementation, provided is a sterile packaged kit of neurovascular tapered catheters. The kit includes a first tapered catheter having a proximal section and a distal tip having a tapered portion. The proximal section is capable of advancing the first tapered catheter through a patient's vasculature to a narrowed portion of a blood vessel. The distal tip has a size and a flexibility to reach the narrowed portion. The kit includes a second tapered catheter having a proximal section and a distal tip having a tapered portion. The proximal section is capable of advancing the second tapered catheter through the patient's vasculature to the narrowed portion of the blood vessel. The distal tip has a size and a flexibility to reach the narrowed portion. A maximum outer diameter of the second tapered catheter is greater than a maximum outer diameter of the first tapered catheter so that the distal tip of the first catheter is insertable into the narrowed portion of the blood vessel, advanced to a dilating position, and removed from the narrowed portion, and thereafter the second catheter is insertable into the narrowed portion of the blood vessel and advanced to a dilating position.

In an interrelated implementation, provided is a method for treating a blood vessel that has been narrowed due to atherosclerosis. The method includes navigating a catheter through a patient's vasculature to a narrowed portion of the blood vessel. The catheter has a proximal section and a distal tip having a tapered portion. The proximal section has a cylindrical surface having an outer diameter greater than an outer diameter of the tapered portion of the distal tip. The proximal section is capable of advancing the catheter through the patient's vasculature, and the distal tip has a size and a flexibility to reach the narrowed portion of the blood vessel. The method includes inserting the distal tip of the catheter into the narrowed portion of the blood vessel; advancing the catheter to a dilating position that is within the narrowed portion so that the tapered portion of the distal tip passes at least partially through the narrowed portion to dilate the narrowed portion; and maintaining the catheter in the dilating position within the narrowed portion for a dwell time of at least 20 seconds so that at least a portion of the narrowed portion is less narrow following removal of the catheter than the narrowed portion was prior to inserting the distal tip of the catheter into the narrowed portion. The blood vessel can be of a head or neck of a patient. The blood vessel can be a peripheral blood vessel or a coronary blood vessel. The peripheral blood vessel can be one in an extremity, including a hand or a foot.

In an interrelated implementation, provided is a method of treating atherosclerotic disease including navigating a catheter through a patient's vasculature to a first narrowed portion in a carotid artery. The first narrowed portion in the carotid artery is an occlusion due to carotid artery disease. The catheter has a proximal section and a distal tip having a tapered portion. The proximal section has a cylindrical surface having an outer diameter greater than an outer diameter of the tapered portion of the distal tip. The proximal section is capable of advancing the catheter through the patient's vasculature. The distal tip has a size and a flexibility to reach the first narrowed portion. The method includes inserting the distal tip of the catheter into the first narrowed portion; advancing the catheter within the first narrowed portion so that the tapered portion of the distal tip passes at least partially through the first narrowed portion to dilate the first narrowed portion; maintaining the catheter in a dilating position within the first narrowed portion for a dwell time selected so that at least a portion of the first narrowed portion is less narrow following removal of the catheter than the first narrowed portion was prior to inserting the distal tip of the catheter into the first narrowed portion; and advancing the catheter towards a second narrowed portion, wherein the second narrowed portion is in an intracranial vessel and the second narrowed portion is due to intracranial atherosclerotic disease.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1A illustrates the anatomy of the human carotid and cerebral blood vessels;

FIG. 1B illustrates is a longitudinal section of a blood vessel having a narrowed portion as a result of atherosclerotic disease;

FIG. 1C is a cross-section taken at arrow C-C of FIG. 1B;

FIG. 2A illustrates an implementation of a catheter for treating a narrowed portion of an atherosclerotic blood vessel having a cylindrical surface along a proximal section and a tapered portion within a distal tip;

FIG. 2B illustrates an implementation of a catheter system incorporating first and second catheters similar to FIG. 2 but having different sized cylindrical surfaces;

FIG. 2C illustrates an implementation of a catheter system incorporating at least one catheter as in FIG. 2A and an outer catheter;

FIG. 2D illustrates an implementation of a catheter system incorporating at least a first and a second catheter as in FIG. 2B and an outer catheter;

FIGS. 3A-3D illustrate implementations of distal tips for the catheter of FIG. 2A;

FIG. 4 illustrates an implementation of a distal tip for the catheter of FIG. 2A;

FIG. 5 illustrates a distal tip of a catheter as in FIG. 2A having a guidewire extending there through;

FIG. 6A illustrates a catheter navigating through a patient's vasculature towards a narrowed portion in a blood vessel due to atherosclerotic disease;

FIG. 6B illustrates a tapered portion of a distal tip of the catheter of FIG. 6A inserting into the narrowed portion of the blood vessel;

FIG. 6C illustrates the tapered portion advanced within the narrowed portion so that the tapered portion of the distal tip passes at least partially through the narrowed portion to dilate the narrowed portion;

FIG. 6D illustrates the cylindrical surface advanced to be within the narrowed portion so that the tapered portion of the distal tip extends distal to the narrowed portion;

FIG. 6E illustrates the catheter withdrawn and the narrowed portion is less narrow following removal of the catheter than the narrowed portion in FIG. 6A;

FIG. 7A illustrates a catheter system having an inner catheter and an outer catheter being advanced through a base sheath positioned in an internal carotid artery towards a narrowed portion in a blood vessel due to atherosclerotic disease;

FIG. 7B illustrates a tapered portion of a distal tip of the inner catheter of FIG. 7A inserting into the narrowed portion of the blood vessel so that the tapered portion of the distal tip passes at least partially through the narrowed portion to dilate the narrowed portion;

FIG. 7C illustrates the outer catheter advanced over the inner catheter of FIG. 7B;

FIG. 7D illustrates the inner catheter withdrawn from the outer catheter inserted through the narrowed portion.

It should be appreciated that the drawings are for example only and are not meant to be to scale. It is to be understood that devices described herein may include features not necessarily depicted in each figure.

### DETAILED DESCRIPTION

There is a need for devices, systems, and methods to safely and quickly access and treat occlusions within blood vessels, including the distal carotid and cerebral arteries, as well as veins, particularly veins in the neck, to restore blood perfusion to and from the brain. For example, there is a need for devices, systems, and methods to treat narrowed portions of a blood vessel due to, for example, atherosclerotic disease, to restore blood perfusion to and from the brain.

Ischemia within the brain may lead to permanent neurologic deficit or death if not quickly resolved. The timeframe for effective treatment of stroke is within 3-4 hours for intravenous (IV) thrombolytic therapy and 6 hours for site-directed intra-arterial thrombolytic therapy or up to 7-8 hours for interventional recanalization of a blocked cerebral artery, and up to 24 hours in certain cases. Re-perfusing the ischemic brain after this time period may not provide benefit to the patient, and may potentially cause harm due to the increase of certain risks. Even within this time period, there is strong evidence that the shorter the time period between onset of symptoms and treatment, the better the results. Unfortunately, the ability to recognize symptoms, deliver patients to stroke treatment centers, and finally to treat these patients within this timeframe is challenging. Despite treatment advances, stroke remains the third leading cause of death and the leading cause of serious, long-term disability in the United States.

Other occlusions can be present, including occlusions that are a result of atherosclerotic disease or atherosclerosis. Atherosclerosis is the build-up of fatty deposits and/or plaque on the inner wall of a patient's arteries. Intracranial vessels including basilar artery, internal carotid arteries ICA, middle cerebral arteries MCA, intracranial vertebral arteries, posterior cerebral arteries, and anterior cerebral arteries ACA can be prone to develop atherosclerosis including intimal necrosis, the accumulation of lipids and development of fatty streaks and fibromuscular plaque, intimal thickening and proliferative changes of the basement membrane and adventitia. Atherosclerotic disease in these vessels is referred to as intracranial atherosclerotic disease (ICAD) and is considered a major cause of recurrent stroke and transient ischemic attacks. Atherosclerotic disease in the arteries of the neck is most commonly called carotid artery stenosis (CAS) or carotid artery disease (CAD) and can also lead to stroke and transient ischemic attacks. Stroke associated with atherosclerotic disease occur most commonly due to artery-to-artery embolism as well as local branch occlusion, in situ thrombotic occlusion, and hemodynamic insufficiency (See Wondwossen et al. Neurology Vol. 97(20 S2): S146-S157 (2021).

The preferred treatment in AIS is removal of the occlusion, whether mechanically or by thrombolytic therapy. For patients with atherosclerotic disease, the occlusion may be treated with catheter-mediated angioplasty, with or without stenting, to open the narrowed portion of the artery resolving the hemodynamic insufficiency, preventing further narrowing and also mitigating the risk of artery-to-artery embolism and stroke associated with atherosclerotic disease.

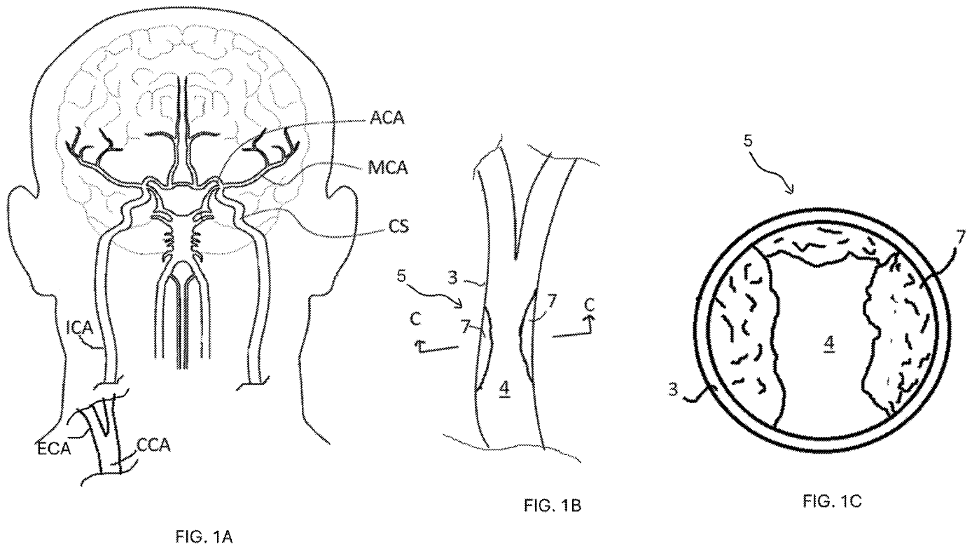

FIG. 1A illustrates the anatomy of the human carotid and cerebral blood vessels showing branching of carotid and cerebral arteries. Veins in the neck are not shown. FIG. 1B is a longitudinal section of a blood vessel 3 in schematic having a narrowed portion 5 as a result of atherosclerotic disease. The occlusion resulting from atherosclerotic disease can include a lipid plaque 7 protruding into the lumen 4 of the blood vessel 3 forming a narrowed portion 5. The occlusion can include additional components, such as a fibrous cap, calcium deposits, and inflammatory cells. The occlusion can include a thrombus that has collected at the plaque 7 with the thrombus and the plaque 7 forming the narrowed portion 5. FIG. 1C is a cross-section of the narrowed portion 5 as a result of atherosclerotic disease showing the lumen 4 of the blood vessel 3 nearly occluded by the plaque 7. The narrowed portion 5 due to the atherosclerotic disease decreases the size of the blood vessel lumen 4 and limits blood flow through the vessel 3.

Navigating the vascular anatomy in order to treat various vascular pathologies in the head and neck at the level of the carotid arteries or cerebral arteries, jugular or cerebral veins, etc., involves use of catheter systems having superior flexibility and deliverability. Again with respect to FIG. 1A, the internal carotid artery ICA arises from the bifurcation of the common carotid artery CCA at the level of the intervertebral disc between C3 and C4 vertebrae. The course of the ICA is divided into four parts—cervical, petrous, cavernous and cerebral parts. In the anterior circulation, the consistent tortuous terminal carotid is locked into its position by bony elements. The cervical carotid enters the petrous bone and is locked into a set of turns as it is encased in bone. The cavernous carotid is an artery that passes through a venous bed, the cavernous sinus, and while flexible, is locked as it exits the cavernous sinus by another bony element, which surrounds and fixes the entry into the cranial cavity. Because of these bony points of fixation, the petrous and cavernous carotid and above are relatively consistent in their tortuosity. The carotid siphon CS is an S-shaped part of the terminal ICA. The carotid siphon CS begins at the posterior bend of the cavernous ICA and ends at the ICA bifurcation into the anterior cerebral artery ACA and middle cerebral artery MCA. The ophthalmic artery arises from the cerebral ICA, which represents a common point of catheter hang up in accessing the anterior circulation. The MCA is initially defined by a single M1 segment and then further bifurcates in two or three M2 segments and then further arborizes to create M3 segments. These points of catheter hang up can significantly increase the amount of time needed to restore blood perfusion to the brain, which in the treatment of atherosclerotic disease, including ICAD, or conditions like AIS that impair blood flow to the brain is a disadvantage with severe consequences.

In addition to the natural anatomy being problematic for navigating catheters to distal sites, the vessels leading to the distal sites can have one or more pathologies that are problematic for advancement of conventional guidewire-led systems. Patients may have an occlusion at a site proximal to the distal treatment site, such as an occlusion within the carotid artery. Patients may also have a severe atherosclerotic disease from a proximal location such as the cervical carotid that can extend through the origin of the ICA as high as the M1 segment. In this situation, the entire region is severely or completely occluded and is often poorly visualized or invisible on angiogram. A severely atherosclerotic vessel may also have a dissection or a breakdown of the layers of the vessel wall. Meaning, a flap of the vessel wall may protrude into the true lumen forming a false lumen. Partial or complete occlusions of the carotid and presence of arterial dissections greatly increases the difficulty in locating and navigating catheters through the anatomy. Conventional guidewire systems tend to go under a dissection flap causing the guidewire to get redirected away from the true lumen of the vessel and into branch vessels as well as greatly increasing the risk of vessel perforation.

With advancing age, the large vessels often enlarge and lengthen. Fixed proximally and distally, the cervical internal carotid artery ICA often becomes tortuous with age. The common carotid artery CCA is relatively fixed in the thoracic cavity as it exits into the cervical area by the clavicle. The external and internal carotid arteries ECA, ICA are not fixed relative to the common carotid artery CCA, and thus they develop tortuosity with advancing age with lengthening of the entire carotid system. This can cause them to elongate and develop kinks and tortuosity or, in worst case, a complete loop or so-called “cervical loop”. If catheters used to cross these kinked or curved areas are too stiff or inflexible, these areas can undergo a straightening that can cause the vessel to wrap around or “barbershop pole” causing focused kinking and folding of the vessel. These sorts of extreme tortuosity also can significantly increase the amount of time needed to restore blood perfusion to the brain, particularly in the aging population. In certain circumstances, the twisting of vessels upon themselves or if the untwisted artery is kinked, normal antegrade flow may be reduced to a standstill creating ischemia. Managing the unkinking or unlooping the vessels such as the cervical ICA can also increase the time it takes to perform a procedure.

Described herein are catheters, catheter systems, and methods of using the catheters and catheter systems for treating various neurovascular pathologies, such as extracranial and intracranial vessel occlusions, caused by atherosclerotic disease. The catheters, catheter systems, and methods described herein restore patency and luminal caliber of the vessels with lower procedural complications (e.g., dissection, perforation, embolism). In addition to treating narrowings of neurovascular vessels, carotid blood vessels, or cerebral blood vessels, the catheters, catheter systems, and methods described herein can be used to treat peripheral blood vessels, coronary blood vessels and other vessels in order to restore blood flow that has been impaired due to any of a variety of conditions, including one or more of peripheral artery disease, coronary artery disease, atherosclerosis, inflammation, and the like.

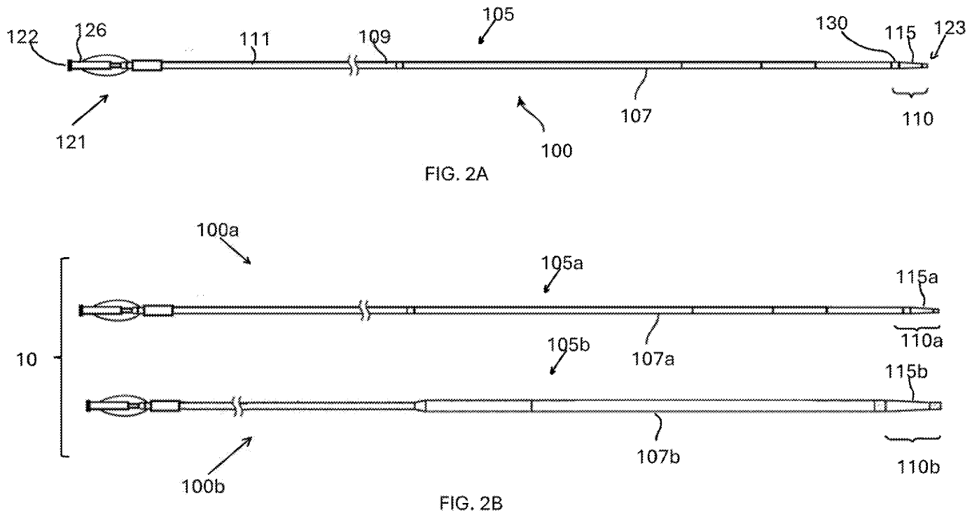

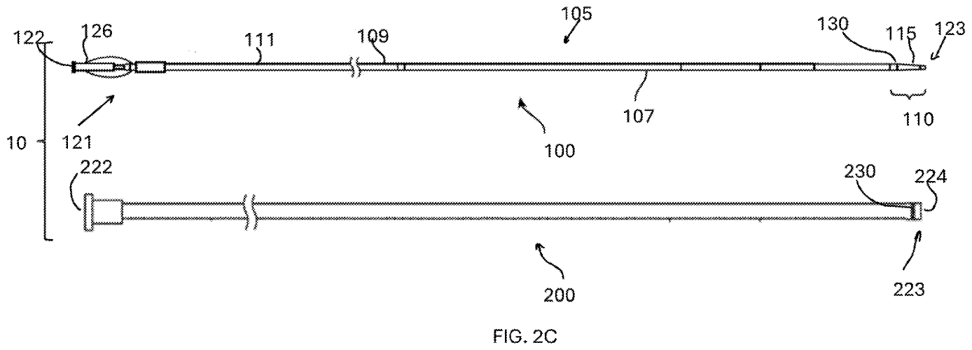

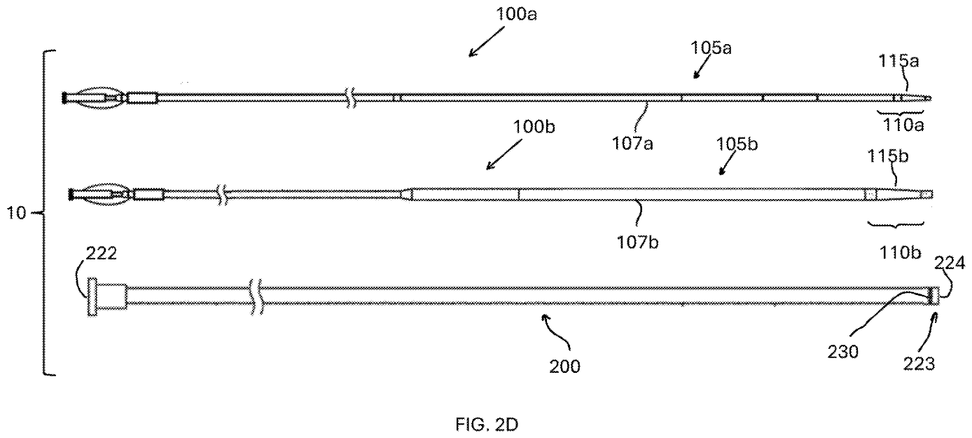

The catheters, catheter systems, and methods include a catheter having a soft, atraumatic, flexible, lubricious distal tip with a tapered portion, such as any of the catheters illustrated in FIGS. 2A-2D, FIGS. 3A-3D, FIG. 4, or FIG. 5 that provide gentle dilation of a narrowing of the vessel and finding the true lumen of the vessel at the narrowing. The catheters, catheter systems, and methods preserve the endothelium of the vessel treatment site thereby lowering the risk of thrombogenicity and re-occlusion. The catheters and catheter systems described herein provide quick and simple access to distal target anatomy, in particular occluded anatomy of extracranial vasculature (e.g., carotid arteries) and the tortuous anatomy of the intracranial vasculature. The catheters and catheter systems described herein are capable of safely navigating to an occlusion, finding the true lumen or path through the occlusion, and dilating the path narrowed by the occlusion despite the navigational challenges of the neurovascular anatomy. The narrowing is widened or made less narrow by the catheters and catheter systems as the tapered catheter passes through the vessel narrowing thereby treating the vessel and optionally allowing for another larger catheter to traverse the narrowing, such as a larger tapered catheter that further widens the narrowing, and/or a corresponding large bore catheter (i.e., aspiration and/or stent delivery system) for delivery to distal sites. The catheters and catheter systems described herein are capable of dilating and increasing the diameter of vessels, such as carotid and cerebral arteries. Widening a narrowed portion or reducing the narrowing of a narrowed portion can include increasing the diameter of a vessel that is narrowed, such as increasing the diameter to a non-symptomatic diameter of the vessel.

The catheters and catheter systems described herein can be used for treatment of a blood vessel narrowing as a result of atherosclerotic disease, including occlusions in carotid and/or cerebral vessels, including the carotid artery, including an internal carotid artery, or a cerebral artery, including a middle cerebral artery (e.g., M1 or M2 segment), and occlusions in jugular and/or cerebral veins. While the catheters and catheter systems described herein are particularly designed to access and treat the neurovasculature (i.e., arteries and veins of the head and those in the neck leading to them), other treatment sites are considered herein including peripheral sites including extremities, such as the hands and feet.

The catheters and catheter systems described herein also enable safe and rapid positioning of working devices configured to provide thrombotic treatments, including large-bore and super-bore aspiration catheters or stent delivery catheters, to an occlusion or narrowing in carotid or cerebral vessel. The shelf-reducing distal tip of the catheters with a tapered portion has a flexibility, taper shape, taper length, wall thickness, etc. for atraumatic delivery to a vessel in the brain that avoids getting misdirected or hung up or, if more force is applied, avoids perforating the vessel. The catheters, catheter systems, and methods need not necessarily incorporate additional interventional devices and procedures, such as stent retrievers and similar devices, which can damage delicate endothelium of the vessel and increase potential for thrombogenicity and re-occlusion. The catheter and catheter systems described herein can be used alone to treat a narrowed portion formed as a result of an occlusion by dilating the narrowed portion. The dilating of the narrowed portion can be performed without treatment of an occlusion distal to the site of the narrowed portion. The narrowed portion need not necessarily be stented or aspirated during or after dilation is performed. The dilation can be performed alone without aspiration, including aspiration thrombectomy. This may be particularly useful where the angiogram demonstrates the narrowed portion is due to an occlusion that is a partial occlusion and a path through the occlusion exists. The aspiration delivered can aspirate in situ thrombus to ‘uncover’ or reveal any underlying ICAD plaque and/or rule out that the occlusion is an embolus. First-pass dilation with a tapered catheter is preferred where an angiogram demonstrates a partial occlusion with ICAD-related vessel changes. The dilation, with or without aspiration, can be performed with or without stenting. A surgeon may choose to forego stenting in an occlusion that is particularly thrombotic, particularly if the occlusion responds well to dilation and stays open following removal of the catheter that performed the dilating. If the narrowed portion does not stay open, a stent may be deployed following dilation of the narrowed portion. As such, the treatment can include dilating the narrowed portion followed by stenting of the dilated narrowed portion. The treatment can include dilating the narrowed portion, with or without stenting of the dilated narrowed portion, with or without balloon angioplasty of the dilated narrowed portion. The treatment can include dilating the narrowed portion, with or without stenting of the dilated narrowed portion, with or without balloon angioplasty of the dilated narrowed portion, in combination with treating an occlusion located distal to the narrowed portion, such as one formed by a distal occlusion that causes acute ischemic stroke. Stenting of the dilated narrowed portion can be performed before or after the distal occlusion is treated. The treatment of the distal occlusion can include delivering a working device, such as an aspiration catheter to aspirate the distal occlusion. Delivery of the working device(s) to a distal occlusion can be aided by the catheter used to initially treat the narrowed portion due to a proximal occlusion. For example, the aspiration catheter can be delivered to the distal occlusion with the aid of the catheter that performed the dilating of the narrowed portion or a secondary catheter larger than the catheter that further dilates the narrowed portion as the secondary catheter is used to advance the aspiration catheter. The treatments of the narrowed portion described herein need not also include treatments of a distal occlusion.

The catheters and catheter systems will be described in more detail below as will the methods of using the catheters and catheter systems to treat the narrowed portion of the blood vessels.

As used herein, “a narrowing” or “a narrowed portion” refers to a location within a vessel that has decreased patency sufficient to disrupt blood flow through the vessel. A narrowing or a narrowed portion can occur as a result of an embolus that causes stroke, atherosclerotic disease, stenosis, and/or as a result of another occlusion.

As used herein, “an occlusion” refers to a narrowing of a blood vessel lumen that results from atherosclerotic disease or stenosis, and/or a narrowing of a blood vessel lumen that resulted from an embolus that lodged within the blood vessel and causes stroke, which can include thrombus that accumulates in situ at the site of the embolus. As used herein “lesion” or “atherosclerotic lesion” or “plaque” or “atherosclerotic plaque” describes the site within a blood vessel in which one or more abnormal histological changes occur due to atherosclerotic disease that contribute to an occlusion in a blood vessel, including one or more of accumulation of atherogenic lipoproteins, immune cells, fatty streaks, extracellular lipid droplets and particles, lipid cores, fibrous connective tissue, the presence of intimal thickening, fissure, hematoma, thrombus, calcium deposits and the like characteristic of various stages of atherosclerotic disease. An “occlusion site” refers to a location within a blood vessel at or near which an occlusion has occurred. An “occlusive material” refers to a material or substance that contributes to or makes up an occlusion, including material contributing to atherosclerosis disease.

As used herein, “embolus” or “embolus material” or “embolic material” or “embolic region” and the like refers to material, which can be occlusive material, within an occlusion site that is a relatively hard or dense consistency. The embolus may be a thrombus (a clot of blood) or other material that formed at a first blood vessel location (e.g., a coronary vessel), broke loose, and traveled through the circulation to a second blood vessel location to create a blood vessel occlusion. An embolus may also be an air bubble, small fatty deposit, or other object carried within the bloodstream to a distant site.

As used herein, “in situ thrombus” “in situ clot material” refers to material, which can be occlusive material, within an occlusion site that accumulates in situ, such as at the site of an embolus, and is often less dense or relatively soft and fluid-like compared to the embolus itself or organized material of the occlusion.

As used herein, “dilating” or “dilation” or “vessel dilation” or “lumen dilation” refers to making a lumen of a narrowed portion of a vessel less narrow or reducing the narrowing of the lumen, such as by widening an occluded true lumen through the vessel to increase the lumen size or substantially return the lumen size back to a size if the vessel did not have the narrowing. Vessel dilation includes compressing an occlusion against a vessel wall to reduce a narrowing of the vessel. Vessel dilation includes widening a narrowed portion or reducing the narrowing of a narrowed portion to increase the diameter of a vessel that is narrowed, such as increasing the diameter to a non-symptomatic diameter of the vessel.

As used herein, “non-expandable” refers to a catheter portion or a surface of a catheter portion that is designed to substantially maintain the same outer diameter during delivery and use, including during dilating of a narrowed portion of a vessel. As an example, a non-expandable catheter or a non-expandable surface of a catheter cannot be actively expanded from a smaller outer diameter to a larger outer diameter, such as by injecting a liquid through an inflation lumen of the catheter causing the surface to project outward. This is in contrast to a balloon catheter having an expandable surface, like a balloon, that is designed to expand from a smaller outer diameter suitable for advancing through a vessel to a larger outer diameter suitable for angioplasty and/or deploying a stent upon injecting a liquid through an inflation lumen. This is also in contrast to stent-like devices or other expandable structures that are designed to expand from a smaller outer diameter suitable for delivery through a vessel to a larger outer diameter in a deployed state.

While some implementations are described herein with specific regard to accessing a neurovascular anatomy for dilation of a narrowing alone or with application of aspiration, the systems and methods described herein should not be limited to this and may also be applicable to other uses. For example, the catheters and catheter systems described herein may be used to prepare a narrowing, such as due to atherosclerotic disease, for the delivery of working devices, such as a stent to the carotid artery or intracranial artery. Where the phrase “distal access catheter” or “aspiration catheter” is used herein that the catheter can be used for aspiration, the delivery of fluids to a treatment site or as a support catheter, or distal access providing a conduit that facilitates and guides the delivery or exchange of other devices such as a guidewire or interventional devices such as stents.

The devices and systems described herein are related to and can be used in combination and in the alternative with the devices and systems described in U.S. Pat. No. 10,327,790, filed Aug. 3, 2012; U.S. Pat. No. 9,561,345, filed Dec. 19, 2014; U.S. Pat. No. 9,820,761, filed Feb. 4, 2016; U.S. Pat. No. 11,020,133, filed on Jan. 9, 2018; U.S. Pat. No. 10,799,663, filed on Jan. 19, 2018; U.S. Publication No. 2019/0351182, filed May 16, 2019; U.S. application Ser. No. 16/684,324, filed Nov. 14, 2019; and U.S. Publication No. 2020/0289136, filed Jun. 2, 2020. The disclosures of each of these publications and applications are incorporated by reference herein in their entireties.

### Catheters and Catheter Systems

FIG. 2A illustrates an implementation of a catheter 100 (referred to herein as a tapered catheter, an inner catheter, or simply a catheter) for navigating through a patient's vasculature and treating a blood vessel (e.g., carotid, cerebral arteries, veins of the neck and head) that is narrowed by atherosclerotic disease by dilating the narrowed portion. Briefly, the tapered catheter 100 includes a proximal section 105 and a distal tip 110 having a tapered portion 115. The proximal section 105 extends proximally to a proximal end 121 of the tapered catheter 100 and the distal tip 110 extends distally to a distal end 123 of the tapered catheter 100. The distal tip 110 and the proximal section 105 of the tapered catheter 100, in the version shown, are non-expandable and have a single lumen 120 (i.e., no more than one lumen) extending therethrough. The non-expandable catheter 100 provides a dilating effect of narrowed portions of a vessel in a manner that is less traumatic compared to, for example, an angioplasty balloon that can be technically challenging to deliver and carries a high risk for vessel dissection, vessel rupture, and thromboembolic complications. The catheter 100 provides a more controlled, lower-profile, and a safer way to mechanically dilate narrowed portions of vessels compared to balloons or other mechanical treatments like stent retrievers, which are limited to treating sites at the locus of the expandable section and only when actively deployed.

The proximal section 105 is used to advance the tapered catheter 100 through the patient's vasculature. The proximal section 105 can include a tubular portion 109 and a proximal extension 111 that is designed to be less flexible than the tubular portion 109 of the proximal section 105. The proximal extension 111 of the proximal section 105 can provide a relatively stiff portion of the tapered catheter 100 suitable for manipulating the more distal regions of the tapered catheter 100. The distal tip 110 has a size and a flexibility to reach the blood vessel having the narrowed portion. The distal tip 110 can be inserted into the narrowed portion so that the tapered portion 115 passes at least partially through the narrowed portion. Moving proximally along a length of the tapered catheter 100, an outer diameter of the atraumatic tapered portion 115 progressively enlarges to an outer diameter of a cylindrical surface 107 of the proximal section 105 thereby dilating the narrowed portion, the cylindrical surface 107 being non-expandable. As will be discussed in more detail herein, the distal tip 110 with the tapered portion 115 atraumatically probes the narrowed portion in a vessel. The smooth, relatively rounded shape having a low friction outer surface tends to encourage deflection of the distal tip 110 relative to the narrowing so that the distal tip 110 finds a passage through it. The tapered portion 115 dilates the narrowed passage as it advanced into/through the passage. Where no passage exists through the narrowing, such as through a complete occlusion, the distal tip 110 atraumatically seats between at least a portion of the occlusion and the vessel wall without risk of dissection or perforation. The tapered catheter 100 and its components will be described in greater detail below.

FIG. 2B illustrates an implementation of a catheter system 10 that includes two tapered catheters 100a, 100b similar in overall structure to one another and to the tapered catheter 100 described above with respect to FIG. 2A. A first tapered catheter 100a of the catheter system 10 has a cylindrical surface 107a of its proximal section 105a with an outer diameter that is different from a corresponding cylindrical surface 107b of the proximal section 105b of the second tapered catheter 100b. The tapered catheters 100a, 100b are sometimes referred to herein as an “inner catheter,” for example, where the tapered catheter is inserted through another catheter, such as a size-matched outer catheter 200 illustrated in the catheter system 10 of FIG. 2C. FIG. 2D illustrates another catheter system 10 having a first tapered catheter 100a and a second tapered catheter 100b where the second tapered catheter 100b is larger than the first tapered catheter 100a and the second tapered catheter 100b is sized-matched to an outer catheter 200. The catheter systems 10 and their components will be described in greater detail below.

Again with respect to FIGS. 2A-2D, the distal tip 110 of the tapered catheter 100 has a design that ensures the distal tip 110 will deflect relative to an organized structure, such as an occlusion due to atherosclerotic disease or an embolus, to find a true lumen or path through a vessel when encountering it within the vessel rather than penetrating the organized structure thereby generating a new lumen or path through the vessel. The distal tip 110 deflects into the path or space whether that space is merely a space between the proximal face of the occlusion and the vessel wall or a narrowed path through the vessel. This is achieved by having a sufficient degree of flexibility of the fully polymeric distal tip 110 so that the tip region deflects readily upon coming into contact with the obstruction (e.g., proximal face of an embolus, an occlusion due to atherosclerotic disease, or dissection flap). The material properties of the distal tip 110 in combination with taper shape, taper length, taper angle, and wall thickness of the distal tip 110 contributes to the overall maximum flexibility that results in the distal tip 110 passing through less organized or less dense regions of a narrowing (e.g., thrombotic material) until the tip encounters a denser structure, such as the vessel wall. The distal tip 110 then deflects away from the organized or dense portion so that, for example, it finds the narrowed path through the atherosclerotic blood vessel. The design of the distal tip 100 finds the path of least resistance in an atraumatic manner without being so flexible or prone to bending that it folds over onto itself and cannot be advanced. The shape of the distal tip 110 allows the tip to insert at least in part within the narrowed path thereby enlarging the narrowed path to a greater inner diameter as it is advanced. The narrowed path through the atherosclerotic disease that is widened by the tapered catheter 100 can be through the central lumen of the vessel surrounded by atherosclerotic plaque on all sides. The narrowed path through the atherosclerotic disease that is widened by the tapered catheter 100 may also include a region that is not surrounded on all sides by atherosclerotic plaque and can be a path between the plaque on just one side of the vessel wall and another side of the vessel wall having no plaque.

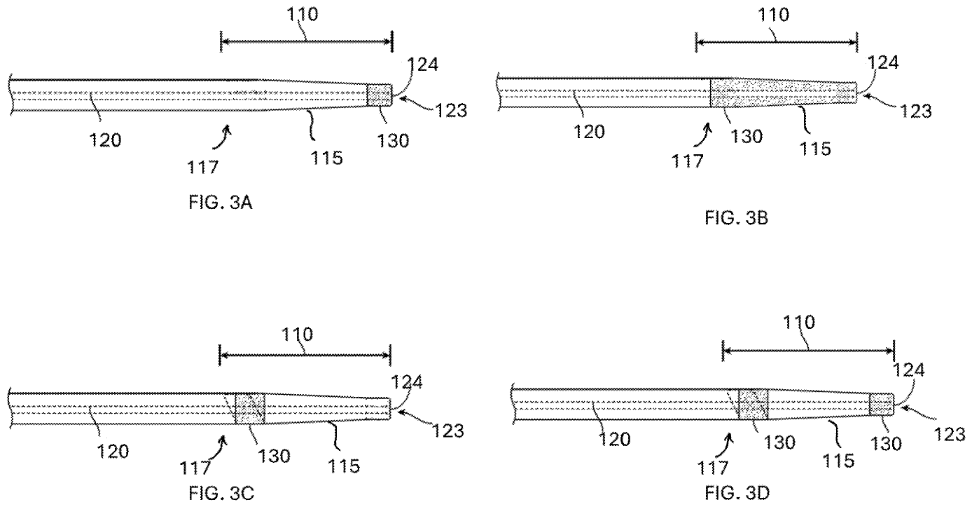

Still with respect to FIGS. 2A-2D and also FIGS. 3A-3D, the distal tip 110 of the tapered catheter 100 can be a substantially non-reinforced, fully polymeric body having a proximal end 117 and a distal end 123. The tapered portion 115 of the distal tip 110 provides a gradual change in outer diameter from a first outer diameter distally to a second outer diameter that is smaller than the first outer diameter. The second outer diameter of the tapered portion 115 closer to the distal end 123 of the tapered catheter 100 can be a relatively smaller size (e.g., about 0.026″-0.040″ OD/0.66 mm-1.016 mm) and the first outer diameter of the tapered portion 115 further away from the distal end 123 of the tapered catheter 100 can be a relative larger size (e.g., 0.062″-0.080″ OD/1.575 mm-2.032 mm). For example, the first outer diameter of the tapered portion 115 can be at least about 1.5 times greater than the second outer diameter of the tapered portion 115. The larger outer diameter can be at least about 1.5 times, 2 times, 2.5 times, or about 3 times larger than the smaller outer diameter of the tapered portion 115. The tapered portion 115 can be a constant taper from the larger outer diameter to the smaller outer diameter.

The length of distal tip 110 and the length of the tapered portion 115 of the distal tip 110 can vary. The distal tip 110 can have a length from the proximal end 117 to the distal end 123 (which can be the distal-most end of the tapered catheter 100) that is greater than a length of the tapered portion 115. For example, the distal tip 110 can be about 1 cm to about 5 cm long from the proximal end 117 to the distal end 123 and the tapered portion 115 can be about 0.5 cm to about 4 cm long along a length of the distal tip 110. In some implementations, the length of the distal tip 110 from the proximal end 117 to the distal end 123 can be in a range of between about 0.50 cm to about 4.0 cm, or between about 1.0 cm to about 3.5 cm, or about 1.5 cm to about 3 cm, or between 2.0 and about 2.5 cm. In some implementations, the length of the distal tip 110 varies depending on the outer diameter of the cylindrical surface 107. For example, the length of the distal tip 110 from the proximal end 117 to the distal end 123 can be about 1.2 cm and have an outer diameter at a proximal end region of the tapered portion 115 of about 0.048″/1.2 mm. The length of the distal tip 110 from the proximal end 117 to the distal end 123 can be about 2.0 cm and have an outer diameter at a proximal end region of the tapered portion 115 of about 0.062″/1.6 mm. The length of the distal tip 110 from the proximal end 117 to the distal end 123 can be about 2.5 cm and have an outer diameter at a proximal end region of the tapered portion 115 of about 0.080″/2.03 mm.

The materials used to form the regions of the tapered catheter 100 can include PEBAX (such as PEBAX 77A, 25D, 33D, 35D, 40D, 42D, 46D, 54D, 55D, 69D, 72D, 90D, etc.) and mixtures or blends of PEBAX (such as a mix of 25D and 35D, 25D and 55D, 25D and 72D, 35D and 55D, 35D and 72D, 55D and 72D, where the blend ratios may range from 0.1% up to 50% for each PEBAX durometer). The distal tip 110 can be the most flexible region of the tapered catheter 100 and transition in flexibility moving proximally along the proximal section 105. The distal tip 110 of the tapered catheter 100 can be a substantially non-reinforced, fully polymeric body. The distal tip 110 can be formed of a material having a material hardness of no more than about Shore 35D. The materials of the tapered catheter 100 proximal of the distal tip 110 can be formed of a material having a material hardness that is greater than about Shore 35D, including the material hardnesses described above (e.g., 40D, 55D, 72D, and combinations thereof). For example, the distal tip 110 can be formed of a material having a material hardness (e.g., 62A and 35D) that transitions proximally towards increasingly harder materials having a material hardness (e.g., 55D and 72D) up to the proximal end 121 of the tapered catheter 100. The proximal section 105 can be substantially non-reinforced, fully polymeric body like the distal tip 110. Alternatively, the proximal section 105 can be at least partially reinforced, such as with a stiffer polymer or a metal braid, coil, and/or hypotube. The metal reinforcement is preferably coated by a polymer.

A lubricious additive compound, such as Mobilize (Compounding Solutions, Lewiston, Maine), can be incorporated into one or more regions. In some implementations, the material used to form a region of the tapered catheter 100 can be Tecothane 62A. Incorporation of a lubricious additive directly into the polymer body of the tapered catheter 100 allows for the tapered catheter 100 to be built without a separate lubricious liner, such as a Teflon liner. This allows for a more flexible element that can navigate the distal cerebral anatomy and is less likely to kink. The tapered catheter 100 can incorporate a separate lubricious liner, such as a liner of PTFE.

The taper shape and taper length and wall thickness of the tapered portion 115 along the fully polymeric, unreinforced distal tip structure allows for the tapered catheter 100 to gently dilate a narrowing due to atherosclerotic disease. The profile of the distal tip 110 of the tapered catheter 100 including the relatively short taper between the outer diameter at the distal end 123 to the larger outer diameter more proximally and the high flexibility of the distal tip 110 results in the tapered catheter 100 deflecting away from organized material encountered at a blood vessel narrowing rather than the distal tip 110 penetrating through it. The tapered catheter 100 probes and deflects away from the narrowing to find a space. For example, a partially occluded, narrowed vessel may still have a lumen extending through it, but the lumen is narrowed so that it is only 2% to 20% patent (see FIGS. 1B-1C). The tapered catheter 100 can deflect away from the more organized portions of the occlusion and atraumatically probe the narrowed lumen through the occlusion finding the space for advancement of the catheter system.

Bending stiffness of the tapered catheter 100 (in N/mm²) can be measured by assessing the bending force in Newtons (N) generated upon deflecting the device a certain distance using a particular gauge length. The bending stiffness (Elastic modulus×area moment of inertia) can be calculated according to the equation EI=FL³/3δ, where F is deflection force, L is gauge length, and δ is deflection. The testing system can include a pin forming a fixed point, an anvil connected by a lever to a strain gauge forming a gauge point. The pin can hold the tapered catheter to be tested such that a gauge length of the tapered catheter is exposed. The anvil is attached to the strain gauge via the lever and can be urged against a portion of the tapered catheter that is positioned away from the pin by the gauge length. The anvil can displace this portion such that the portion triggers a force measurable by the strain gauge. The gauge length can be about 5 mm. The anvil can have a width, for example, about 2 mm, resulting in a minimum gauge length of about 3 mm. The length can vary depending on the testing system.

Table 1 below illustrates various parameters for the atraumatic distal tip 110 of various sized tapered catheters using a 3 mm gauge length (L=3 mm) and deflecting a tip of the catheter 2 mm (δ=2 mm), and generating about 0.05-0.5 N of force. The distal-most bending force was measured at no greater than about 5 mm from the distal-most end of the catheter.

TABLE 1

| | | | | | | Annular distal- | | |
|---|---|---|---|---|---|---|---|---|
| | | | OD1 to | | | facing contact | Force | |
| | | | OD2 | | Max | area (lumen | per unit | Distal-most |
| | OD1 | OD2 | distance | ID | Shore | filled) | area | bending force |
| | (mm) | (mm) | (cm) | (mm) | Hardness | (mm²) | (N/mm²) | (N) |
| X- | 0.74 | 1.0 | 0.9 | 0.4 | 35 D | 0.43 | 2.3 | 0.037 |
| Small | | | | | | | | |
| Small | 0.79 | 1.2 | 1.2 | 0.4 | 35 D | 0.49 | 2.0 | 0.026 |
| Med | 0.79 | 1.6 | 2.0 | 0.5 | 35 D | 0.49 | 2.0 | 0.030 |
| Large | 0.79 | 2.0 | 2.5 | 0.5 | 35 D | 0.49 | 2.0 | 0.021 |

The bending force of the tapered catheter 100 can change over a length of the tapered catheter 100, the bending force being the lowest along the distal tip 110 and the bending force increasing moving proximally towards the proximal end 121 of the tapered catheter 100. The distal-most bending force of the distal tip 110 of the tapered catheters 100 described herein, upon 2 mm deflection and a force gauge length of 3 mm, can be no greater than 0.10 N or between 0.02 and 0.10 N.

The distal-most bending force of the tapered tip having a length that is about 0.5 cm-2.5 cm can be about 0.015-0.045 N. In an implementation, the taper length is about 0.5-1.0 cm from a proximal outer diameter of about 1 mm to a distal outer diameter of about 0.75 mm (taper angle of about 0.72-1.4) and has a distal-most bending force in a range of about 0.029-0.045 N. In another implementation, the taper length is about 1.0-1.5 cm from a proximal outer diameter of about 1.2 mm to a distal outer diameter of about 0.80 mm (taper angle of about 0.8-1.1) and has a distal-most bending force in a range of about 0.023-0.031 N. In another implementation, the taper length is about 1.5-2.0 cm from a proximal outer diameter of about 1.6 mm to a distal outer diameter of about 0.80 mm (taper angle of about 1.1-1.5) and has a distal-most bending force in a range of about 0.020-0.040 N. In another implementation, the taper length is about 2.0-2.5 cm from a proximal outer diameter of about 2.0 mm to a distal outer diameter of about 0.80 mm (taper angle of about 1.4-1.7) and has a distal-most bending force in a range of about 0.017-0.033 N.

The tapered catheter 100 can have an atraumatic distal tip having a bending force that is no greater than about 0.1 N, no greater than about 0.09 N, no greater than about 0.08 N, no greater than about 0.07 N, no greater than about 0.06 N, no greater than about 0.05 N, no greater than about 0.04 N, no greater than about 0.03 N, down to about 0.020 N.

The force per unit area for the tapered catheter 100 is relatively low. For example, the outer diameter of the distal end of the tapered catheter 100 can be about 0.031″ (0.79 mm) and the distal-facing contact area for the atraumatic distal tip of the tapered catheter 100 can be about 8.00×10⁻⁴ square inch (0.5 mm²) if the lumen 120 is filled (e.g., with a column of fluid and/or a guidewire within the lumen). The distal-facing contact area for the atraumatic distal tip of the tapered catheter 100 can be about 4.20×10⁻⁴ square inch (0.27 mm²) for just the annular distal-facing surface without a column of fluid or guidewire within the lumen although can vary depending on the overall size of the catheter 100. The inner diameter of the lumen for the smallest size cylindrical surface 107 can be smaller (about 0.016″/0.4 mm) compared to the inner diameter of the lumen for the larger size cylindrical surfaces 107 (0.019″/0.5 mm). The force per unit area of the tapered catheter 100 for 1 N force is about 1,300 N/square inch (2 N/mm²) to about 2,400 N/square inch (4 N/mm²). The force per unit area of the tapered catheter 100 regardless the maximum outer diameter of the cylindrical surface 107 can be less than about 10 N/mm² or less than about 5 N/mm², or about 2-4 N/mm².

Again with respect to the figures, the tapered catheter 100 can have one or more radiopaque markers 130 along its length. The one or more markers 130 can vary in size, shape, and location. The tapered catheter 100 can incorporate one or more markers 130 along the distal tip 110 (see FIGS. 3A-3D). FIG. 3A illustrates a single marker 130 positioned on the distal tip 110 to indicate the distal end 123 of the tapered catheter 100. FIG. 3B illustrates another implementation having a single marker 130 positioned on the distal tip 110. The single marker 130 indicates the distal end 123 of the tapered catheter 100 and a proximal end of the tapered portion 115. FIG. 3C illustrates a single marker 130 positioned on the distal tip 110 to indicate the proximal end of the tapered portion 115 of the distal tip 110. FIG. 3D illustrates an implementation having two markers 130 including a first marker 130 positioned on the distal tip 110 to indicate the distal end 123 of the tapered catheter 100 and a second marker 130 positioned on the distal tip 110 to indicate the proximal end of the tapered portion 115 of the distal tip 110. The marker can be located to indicate the border between the tapering of the distal tip 110 and the proximal section 105 having a cylindrical surface 107 with a uniform or maximum outer diameter. The marker 130 can also be entirely on the cylindrical portion 107 of the tapered catheter 100 proximal to the distal tip 110 to designate the cylindrical portion 107 of the tapered catheter 100 to be placed at the location of the vessel narrowing throughout the dwell time. It should be appreciated that a single marker can identify both the distal end region and the proximal end of the taper. Identifying a proximal end of the taper provides a user with information regarding an optimal extension of the distal tip 110 relative to a distal end of another catheter, such as the outer catheter 200, to minimize the lip at this distal end for advancement through tortuous anatomy.

The marker material may be a platinum/iridium band, a tungsten, platinum, or tantalum-impregnated polymer, a radiopaque coil embedded in the polymer wall of the catheter, or other radiopaque marker that does not significantly impact the flexibility of the distal tip 110. In some implementations, the radiopaque markers are extruded PEBAX loaded with tungsten for radiopacity. In some implementations, the markers located on different regions of the tapered catheter 100 can have differences in shape, construction, or properties providing discernable information on angiogram, such as a first marker that is a band having one width and a second marker that is a band having a different width.

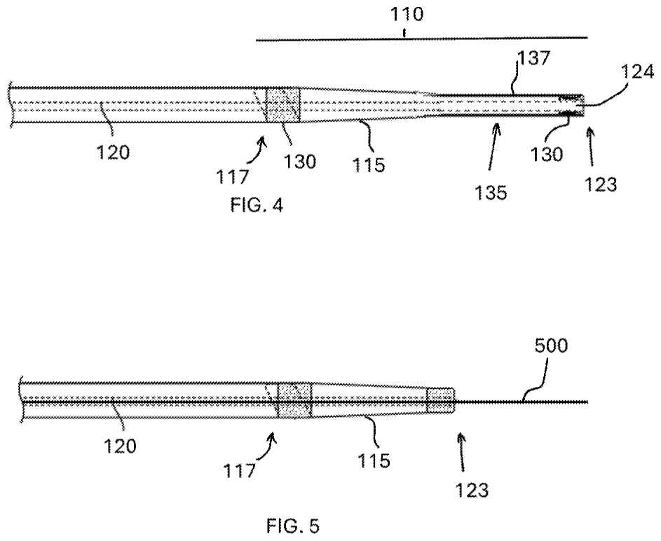

The tapered portion 115 of the distal tip 110 can taper to the distal opening 124 at the distal end 123 of the tapered catheter 100. Alternatively, the tapered portion 115 of the distal tip 110 can taper from a first outer diameter to a second, smaller outer diameter without tapering clear to the distal opening 124 at the distal end 123 of the tapered catheter 100. For example, the distal tip 110 can incorporate an elongated, cylindrical tubular extension 135 that is located distal to the tapered portion 115 as shown in FIG. 4. The tubular extension 135 can have a cylindrical surface 137 that extends from the distal end of the tapered portion 115 to the distal end 123 of the catheter 100. The cylindrical surface 137 of the tubular extension 135 has an outer diameter that is smaller than the outer diameter of the cylindrical surface 107 of the proximal section 105. Like the distal tip 110 without the tubular extension, the distal tip 110 with the tubular extension 135 is designed to smoothly track through the vasculature so as to deflect to find a path through a narrowed portion of a vessel. The distal tip 110 of the tapered catheter 100 can include at least one, at least two, or more markers 130 to identify one or more regions of the tapered catheter 100. FIG. 4 shows the tapered catheter 100 with the tubular extension 135 incorporates a first marker 130 near the distal opening 124 to identify the distal end 123 of the tapered catheter 100 and a second marker 130 near the proximal end 117 of the tapered portion 115 to identify a proximal end region of the taper. The tapered catheter 100 with the tubular extension 135 can additionally incorporate a third marker 130 near the border between the distal end of the tapered portion 115 and the proximal end of the tubular extension 135.

Again with respect to FIGS. 2A-2D, the cylindrical surface 107 of the proximal section 105 has an outer diameter, which can be larger than the outer diameter(s) of the distal tip 110. The outer diameter of the cylindrical surface 107 of the proximal section 105 can be selected in relation to a characteristic of the narrowing being treated and/or the blood vessel being treated. In some implementations, the characteristic is a diameter of the blood vessel to be treated, where the diameter is the size of the blood vessel without the narrowing. As an example, the average diameter of a cervical segment of the internal carotid artery without any significant narrowing due to an occlusion, such as an embolus that causes stroke or atherosclerotic disease, can be about 4-5.4 mm, the diameter of a communicating segment of the internal carotid artery without any significant narrowing can be about 2.8-4 mm, and the diameter of an M1 segment vessel without any significant narrowing can be about 2-3 mm. The outer diameter of the cylindrical surface 107 of the proximal section 105 selected for treating a narrowing of these blood vessel segments can be selected based on the lumenal diameter of the vessel segment when not narrowed as assess, for example, by baseline angiogram, so that a maximum widening of the vessel lumen is achieved by the cylindrical surface 107 without stretching the vessel wall. Vessel wall injury due to stretch can lead to detrimental vessel wall remodeling and restenosis. Thus, the outer diameter of the cylindrical surface 107 selected can be the same as or slightly smaller than the lumenal diameter of the blood vessel when not narrowed to achieve a lumen dilation effect without vessel wall damage. The outer diameter of the cylindrical surface 107 selected can be greater than the lumenal diameter of the blood vessel when not narrowed so that some stretching of the vessel wall is achieved. The outer diameter of the cylindrical surface 107 selected can be greater than a diameter of the blood vessel when narrowed, but less than a diameter of the blood vessel when not narrowed, such that the narrowing itself is widened by the outer diameter of the cylindrical surface 107 without stretching the vessel wall itself.

The outer diameter of the cylindrical surface 107 selected can be additionally or alternatively in relation to the type of occlusion being treated by the tapered catheter 100. Similarly, the dwell time can vary depending on the outer diameter of the cylindrical surface 107 selected. For example, the occlusion can be a partial occlusion with at least some flow through the lumen of the vessel and the occlusion can be compressed against the vessel wall by the cylindrical surface 107 to increase lumen size. A larger sized outer diameter may be selected to perform the dilating of the partial occlusion for a shorter dwell time compared to a dwell time selected for a smaller sized outer diameter.

The occlusion can be due to atherosclerotic disease, which can be characterized by the presence of stable or vulnerable plaques or lesions causing the lumen of the blood vessel to narrow. Stable atherosclerotic lesions may be associated with a thick fibrous cap, microcalcification and extensive fibrous tissue whereas vulnerable atherosclerotic lesions may be associated with a large necrotic core, a thin fibrous cap, inflammation and macrophage infiltration), angiogenesis, plaque hemorrhage, and microcalcification. The tapered catheter size, dwell time, and/or number of reinsertions selected can be based on these differences. A larger outer diameter can be selected for a narrowing caused by a vulnerable atherosclerotic lesion and/or a shorter dwell time and/or number of reinsertions compared to an occlusion caused by a stable atherosclerotic lesion, and the like.

Advancing a catheter into, within, or through a narrowed portion of a vessel to be treated can be performed relatively slowly. For example, a velocity of the catheter being advanced into, within, or through the narrowed portion of a vessel to be treated (referred to herein as a “crossing velocity”) can be no more than about 3 cm/second. The crossing velocity can be slower than, for example, the velocity a catheter is passed through vessels that are free of any narrowing. The crossing velocity into, within, or through the narrowed portion ensures optimal dilating force of the narrowed portion is achieved by the maximum outer diameter region of the catheter. The crossing velocity can be about 0.01 cm/second, about 0.05 cm/second, about 0.25 cm/second, about 0.50 cm/second, about 0.75 cm/second, up to about 1 cm/second and anywhere in between. The crossing velocity can be no more than about 1 cm/second, no more than about 1.5 cm/second, no more than about 2 cm/second, no more than about 2.5 cm/second, or no more than about 3 cm/second and anywhere in between. The crossing velocity can be slower for catheters having a cylindrical surface 107 with the maximum OD that is shorter and faster for catheters having a cylindrical surface 107 with the maximum OD that is longer. For example, where the large diameter region of the catheter is shorter, the catheter can be advanced to cross through the narrowed portion more slowly to achieve an effective “dwell” of the narrowing as the shorter, large diameter region of the catheter passes through. Where the large diameter region of the catheter is longer, the catheter can be advanced to cross through the narrowed portion more quickly because the effective “dwell” of the narrowing as the longer, large diameter region of the catheter passes through is extended. In some implementations, the maximum outer diameter region of the cylindrical surface 107 is very long (e.g., nearly an entire working length of the catheter) and the crossing velocity for such catheters can be to ensure the dilating is most effective for the narrowed portion being treated.

A primary efficacy outcome of the treatments described herein is successful reperfusion on a final angiogram following treatment and can be defined as expanded in comparison to measurement of the degree of narrowing before treatment. The goal of endovascular treatments for acute ischemic stroke of the anterior circulation according to the current American Heart Association guidelines can be scored according to the Expanded Thrombolysis in Cerebral Infarction (eTICI) scale (see, e.g., LeCouffe et al. Stroke 2020; 51:1790-1796). The degree of reperfusion can be assessed and scored based on this scale.

Degree of narrowing can be assessed by non-contrast computed tomography (CT), CT angiography, CT perfusion, magnetic resonance angiography, and procedural digital subtraction angiography (DSA) images, and the like. ICAD can be defined by angiographic evidence of an occlusion, such as a tapered-shape occlusion, presence of arterial calcific atherosclerotic lesions, remnant stenosis greater than about 70% after initial aspiration thrombectomy. Other angiographic features, such as the presence of intracranial carotid artery atherosclerotic changes, presence of multifocal narrowing and/or the presence of leptomeningeal collateral. Post-treatment angiography can be performed over a period after withdrawal of the tapered catheter 100 to evaluate patency, re-thrombosis, recoil, or re-occlusion. Additional rescue therapy, such as with ancillary devices, such as balloon angioplasty and/or stenting is considered herein.

Insertion of the outer diameter of the cylindrical surface 107 of the proximal section 105 into the narrowed portion of the vessel provides a maximum dilation of the narrowed portion provided by the tapered catheter 100 and an increased hardness and/or stiffness relative to the distal tip 110 to further facilitate dilation. The proximal section 105 can be relatively uniform in outer diameter extending along at least a portion of its length. The proximal section 105 having the cylindrical surface 107 with the larger outer diameter (e.g., 0.048″-0.080″) preferably extends along a length that is at least about 5 cm, at least about 10 cm, at least about 20 cm, or at least about 30 cm or more including up to about the full length of the proximal section 105 without any step down in outer diameter. The length of the cylindrical surface 107 having the larger outer diameter can be at least about 5 cm, at least about 6 cm, at least about 7 cm, at least about 8 cm, at least about 9 cm, at least about 10 cm, at least about 11 cm, or at least about 12 cm up to about 50 cm. The outer diameter of the proximal section 105 also can taper proximally or step down in outer diameter along at least a portion of its length. The transition in outer diameter proximally can incorporate a smooth taper or step change in outer diameter. As mentioned above, the proximal section 105 is used to advance the tapered catheter 100 through the patient's vasculature. The proximal extension 111 of the proximal section 105 can provide a relatively stiff portion of the tapered catheter 100 suitable for manipulating the more distal regions of the tapered catheter 100. For example, the proximal extension 111 can be or can incorporate a metal hypotube, cut hypotube, coil, braid, or other reinforced segment that allows for one-to-one translation of advancement forces and controlled pushability.

The tapered catheter 100 can be of one of a variety of sizes, including extra-small, small, medium, large, and extra-large depending on the needs of the treatment and whether the tapered catheter 100 will be used to deliver another catheter, such as outer catheter 200, or catheter system. As an example, the outer diameter of the cylindrical surface 107 of the proximal section 105 can be a smaller-sized version that is about 0.039 inch (1.0 mm) up to about 0.071 inch (1.8 mm). The outer diameter of the cylindrical surface 107 of the proximal section 105 can be a larger-sized version that is about 0.055 inch (1.4 mm) up to about 0.088 inch (2.2 mm). In some implementations, the tapered portion 115 of the distal tip 110 can be from about 0.039 inch (1.0 mm) outer diameter down to about 0.029 inch (0.740 mm) outer diameter over a length of about 1.0 cm. In some implementations, the tapered portion 115 of the distal tip 110 can be from about 0.048 inch (1.2 mm) outer diameter down to about 0.031 inch (0.787 mm) outer diameter over a length of about 1.2 cm. In some implementations, the tapered portion 115 of the distal tip 110 can be from 0.062 inch (1.575 mm) outer diameter to about 0.031 inch (0.787 mm) outer diameter over a length of about 2 cm. In still further implementations, the tapered portion 115 of the distal tip 110 can be from 0.080 inch (2.032 mm) outer diameter to about 0.031 inch (0.787 mm) outer diameter over a length of about 2.5 cm.

Various combinations of tapered catheter 100 sizes are considered herein and the tapered catheters 100 can be combined into a system of catheters 10 combining the various sizes (e.g., a kit or set of catheters packaged together). The proximal section 105 of a first tapered catheter 100a in the catheter system 10 can have a cylindrical surface 107 that has an outer diameter greater than an outer diameter of the cylindrical surface of the second tapered catheter 100b in the catheter system 10 and also greater than an outer diameter of the tapered portion 115 of the distal tip 110 of the second tapered catheter 100b. In an implementation, the catheter system 10 includes two tapered catheters 100a, 100b in which the first tapered catheter 100a has a cylindrical surface 107 having an outer diameter that is 0.048″/1.2 mm and the second tapered catheter 100b has a cylindrical surface 107 having an outer diameter that is 0.062″/1.6 mm. In another implementation, the catheter system 10 includes two tapered catheters 100a, 100b in which the first tapered catheter 100a has a cylindrical surface 107 having an outer diameter that is 0.048″/1.2 mm and the second tapered catheter 100b has a cylindrical surface 107 having an outer diameter that is 0.080″/2.03 mm. In another implementation, the catheter system 10 includes two tapered catheters 100a, 100b in which the first tapered catheter 100a has a cylindrical surface 107 having an outer diameter that is 0.062″/1.6 mm and the second tapered catheter 100b has a cylindrical surface 107 having an outer diameter that is 0.080″/2.03 mm. In still further implementations, the catheter system 10 includes three tapered catheters 100a, 100b, 100c in which the first tapered catheter 100a has a cylindrical surface 107 having an outer diameter that is 0.048″/1.2 mm and the second tapered catheter 100b has a cylindrical surface 107 having an outer diameter that is 0.062″/1.6 mm and the third tapered catheter 100c has a cylindrical surface 107 having an outer diameter that is 0.080″/2.03 mm. In any of these implementations, the catheter system 10 includes a first tapered catheter 100a that has a cylindrical surface 107 having an outer diameter that is less than 0.048″/1.2 mm and includes a second tapered catheter 100b that has a cylindrical surface 107 having an outer diameter that is greater than or equal to 0.048″/1.2 mm. In any of these implementations, the catheter system 10 includes a first tapered catheter 100a that has a cylindrical surface 107 having an outer diameter that is less than or equal to 0.080″/2.03 mm and includes a second tapered catheter 100b that has a cylindrical surface 107 having an outer diameter that is greater than 0.080″/2.03 mm.

One or more of the tapered catheters 100 of different sizes discussed above can be provided as a kit of catheters.

In a first implementation, the kit includes three sizes of tapered catheters 100a, 100b, 100c in which the first tapered catheter 100a has a cylindrical surface 107 having an outer diameter that is 0.048″/1.2 mm and the second tapered catheter 100b has a cylindrical surface 107 having an outer diameter that is 0.062″/1.6 mm and the third tapered catheter 100c has a cylindrical surface 107 having an outer diameter that is 0.080″/2.03 mm. In a second implementation, the kit includes two sizes of tapered catheters 100a, 100b in which the first tapered catheter 100a has a cylindrical surface 107 having an outer diameter that is 0.048″/1.2 mm and the second tapered catheter 100b has a cylindrical surface 107 having an outer diameter that is 0.062″/1.6 mm. In another implementation, the catheter system 10 includes two tapered catheters 100a, 100b in which the first tapered catheter 100a has a cylindrical surface 107 having an outer diameter that is 0.048″/1.2 mm and the second tapered catheter 100b has a cylindrical surface 107 having an outer diameter that is 0.080″/2.03 mm. In another implementation, the catheter system 10 includes two tapered catheters 100a, 100b in which the first tapered catheter 100a has a cylindrical surface 107 having an outer diameter that is 0.062″/1.6 mm and the second tapered catheter 100b has a cylindrical surface 107 having an outer diameter that is 0.080″/2.03 mm. In a third implementation, the kit includes multiple tapered catheters of different sizes with a single outer catheter. The kits can incorporate one or more additional components in a single box for performing a complete procedure, including one or more of syringe, heparinized saline or other flush solution, one or more rotating hemostatic valves (RHV), guidewire, introducer kit, long sheath, select catheter, dilator, super-bore RHV, connector clip for components, and the like. In another implementation, the kit incorporates all the components for performing an ICAD treatment as discussed herein.

Where the cylindrical surface 107 has an outer diameter that is 0.048″/1.2 mm, 0.062″/1.6 mm, or 0.080″/2.03 mm, the length of the cylindrical surface 107 can be at least 5 cm, at least 10 cm, at least 20 cm, or at least 30 cm or more. Longer lengths are considered, for example, for peripheral disease. The tapered catheters described herein can incorporate a cylindrical surface 107 in these outer diameter ranges (e.g., 0.048″-0.080″) that can be as short as about 5 cm or as long as 30 cm up to the full working length of the tapered catheter and without sacrificing flexibility for navigation to the neurovasculature. This allows for the same sized catheter to be used anywhere to treat any length narrowing. In contrast, balloons of angioplasty balloon catheters must be kept relatively short (e.g., about 1 cm-3 cm long) to maintain sufficient flexibility of the catheter for navigation to distal sites. This is a disadvantage for segments of vessels that are to be treated due to presence of long ICAD. The short balloon of an angioplasty balloon catheter must be repeatedly aligned and deployed along multiple segments of these long treatment sites, which increases complexity of the procedure and time necessary to resolve the narrowing.

As discussed elsewhere herein, the tapered catheter 100 has a non-expandable cylindrical surface 107 proximal of the distal tip 110 and with an outer diameter greater than that of the tapered portion of the distal tip 110 that can be used to perform the dilating of the narrowed portion (e.g., a dilating segment). The dilating segment extends along a length (e.g., a dilating segment length) of the tapered catheter 100. The dilating segment can vary in outer diameter as discussed elsewhere herein (e.g., 0.048″-0.080″). The dilating segment length of the dilating segment can vary as well. In some implementations, the dilating segment length is about 10-30 cm, about 15-25 cm, or about 18-22 cm, and any length in between including 1-30 cm, 10-20 cm, 20-30 cm, for example. The dilating segment length of the dilating segment can incorporate a surface treatment, such as a hydrophilic coating and/or a drug coating as described elsewhere herein.

The dilating segment can be located proximal of the distal tip 110 and distal to the stiffer regions of the tapered catheter 100. The dilating segment of the tapered catheter 100 can be formed of a polymer material having a material hardness that is less than about Shore 72D, less than about Shore 55D, or less than about Shore 45D. The polymer material of the dilating segment can have a material hardness that is the same as the material hardness of the polymer material of the distal tip 110, such as about Shore 35D. The polymer material of the dilating segment can have a material hardness that is the greater than the material hardness of the polymer material of the distal tip 110 and no greater than about Shore 55D. The distal tip 110 that is formed of a material having a material hardness no greater than about Shore 35D can function to probe and find a passage or true lumen through the narrowed portion of the vessel while the dilating segment proximal to the distal tip 110 that is formed of a slightly harder material having a material hardness greater than Shore 35D, such as about Shore 55D, can function to dilate the narrowed portion. It should be appreciated that the Shore hardness values are being used as examples and that variations are considered as well, including blends of materials (e.g., Shore 35D/40D, Shore 35D/55D, etc.). Generally, the dilating segment of the tapered catheter 100 is located proximal of the tapered portion 115 of the distal tip 110, has a relatively large outer diameter sized to perform the dilating, and is formed of a polymer material having a material hardness sufficient to dilate a narrowed portion long-term. The dilating segment can have the same outer diameter as more proximal sections of the tapered catheter 100, such as the proximal section used by a surgeon to advance the catheter through the vessel, but is generally more flexible than those proximal sections so as to take sharp turns (e.g., brachiocephalic take-off, ophthalmic artery take-off) and advance into distal vessels (e.g., middle cerebral arteries) without risk of kinking or ovalizing.

Given the risks of unintended complications with balloon angioplasty, including vessel rupture and dissection, the catheters and catheter systems provide an alternative mechanical therapy (e.g., a modified Dotter technique) involving dilating with a non-expandable tapered catheter and providing a safer alternative.

The lumen 120 of the tapered catheter extends from a proximal opening 122, such as at the proximal end 121 of the tapered catheter 100 configured to remain outside the body during use, to the distal opening 124 at the distal end 123 of the tapered catheter 100. Where the tapered catheter 100 incorporates a tubular extension 135 (see FIG. 4), the lumen 120 extends through the tubular extension 135 to the distal opening 124 from the lumen 120. The lumen 120 is preferably designed for over-the-wire methods although rapid exchange configurations are considered herein as well. The lumen 120 can have a uniform inner diameter from the proximal opening 122 to the distal opening 124 so that the inner diameter of the lumen 120 remains unchanged over the working length of the tapered catheter 100, even through the tapered portion 115 of the distal tip 110 (and the tubular extension 135, if present). The wall thickness of the distal tip can thin distally along the length of the tapered portion 115 providing the change in outer diameter of the taper while maintaining a uniform inner diameter. Alternatively, the inner diameter of the lumen 120 can change over the length of the tapered catheter 100. The inner diameter of the lumen 120 can have a first size through the tapered portion 115 of the distal tip 110 and a second, larger size through the at least a region of the proximal section 105.

The lumen 120 of the tapered catheter 100 can be less than about 0.024″, such as about 0.014″ (0.356 mm)-0.024″ (0.610 mm). The lumen 120 of the tapered catheter 100 can be sized to receive a guidewire in the range of 0.014″ (0.356 mm) and 0.018″ (0.457 mm) diameter. Thus, the tapered catheter 100 has a relatively large outer diameter within the proximal section 105 and a relatively small inner diameter, particularly when a guidewire 500 extends into or through the lumen 120 (see FIG. 5).

Again with respect to FIG. 2A, the tapered catheter 100 can include a proximal luer 126 coupled to the proximal end 121 of the tapered catheter 100. Tools, such as a guidewire 500, can be advanced through the proximal luer 126 into the proximal opening 122 of the lumen 120 of the tapered catheter 100. A syringe or other component can be coupled to the proximal luer 126 in order to draw a vacuum and/or inject fluids through the lumen 120. The syringe coupled to the luer 126 can also be used to close off the lumen 120 of the tapered catheter 100.

The lumen 120 of the tapered catheter 100 is sized to accommodate a guidewire 500. The tapered catheter 100, however, is capable of being used without guidewires. Guidewires are designed to be exceptionally flexible so that they deflect to navigate the severe turns of the anatomy. However, many workhorse guidewires have a stiffness along their longitudinal axis and/or are small enough in outer diameter that they find their own paths through an occlusion rather than slipping around the occlusion. Guidewires also tend to catch on vessel wall dissections increasing the risk of perforations. In some cases, these guidewires can cause perforations and/or dissections of the vessel itself. Guidewires tend to get redirected into branches rather than remaining within the larger vessel. This makes them helpful for selecting a vessel branch, but problematic for navigating tortuous anatomy and following the main flow of blood. Thus, even though the guidewire may have an outer diameter at its distal tip region that is small and very flexible at the distal tip, guidewires typically are incapable of atraumatically probing an occlusion or other structure such that they pose a risk of perforation with repeated advancement. Guidewires may not deflect upon encountering something relatively dense such as the proximal face of the occlusion nor a dissection flap and instead embed and penetrate such structures.

The tapered catheter 100 is designed specifically to have a softness, taper, and sizing that finds and/or creates space, even without a guidewire. For example, the tapered catheter 100 upon encountering a complete vessel occlusion, such as an embolus, can slide between a portion of the embolus and the vessel wall rather than penetrating through it like a guidewire does. In the case of a partially occluded vessel, such as an atherosclerotic narrowing as shown in FIGS. 1B-1C, the tapered catheter 100 can atraumatically and safely find the path through the narrowing thereby dilating the narrowed portion as described elsewhere herein. The softness, taper, and sizing of the catheter 100 allows for it to be repeatedly passed through vessels without penetrating or taking a detour relative to these structures. Methods of using the tapered catheter 100, with or without a guidewire, is described in more detail below.

The overall length of the tapered catheter 100 (e.g., between the proximal end 121 near the luer 126 to the distal end 123) can vary, but generally is long enough to extend to at least an M1 segment of the middle cerebral artery from an access site at the femoral artery or another access site, while at least a length of the proximal section 105 remains outside an access catheter positioned at the arteriotomy outside the body of the patient. The length of the tapered catheter 100 can allow for the distal opening 124 to reach cerebrovascular targets or occlusions within, for example, segments of the internal carotid artery including the cervical (C1), petrous (C2), lacerum (C3), cavernous (C4), clinoid (C5), ophthalmic (C6), and communicating (C7) segments of the internal carotid artery (ICA) as well as branches off these segments including the M1 or M2 segments of the middle cerebral artery (MCA), anterior cerebral artery (ACA), anterior temporal branch (ATB), and/or posterior cerebral artery (PCA). For example, the entry location of the tapered catheter 100 can be in the femoral artery and the target occlusion location can be distal to the right common carotid artery, such as within the M1 segment of the middle cerebral artery on the right side. In some implementations, the overall length of the tapered catheter 100 is about 155 cm-180 cm and has a working length of about 150 cm-170 cm. It should be appreciated that where the tapered catheter 100 is described as accessing or passing through an artery, that the tapered catheter 100 can access or pass through a vein at the corresponding level.

As mentioned above, the tapered catheter 100 having the distal tip 110 with the tapered portion 115 can be used on its own, along with another tapered catheter 100, and/or can be part of a catheter system 10 having a size-matched outer catheter 200 (see FIGS. 2C-2D). The catheter systems 10 described herein can incorporate one or more catheters 100 having a distal tip 110 with a tapered portion 115 (also referred to herein as an “inner catheter”) and one or more catheters 200 (also referred to herein as an “outer catheter”) sized to receive a tapered catheter 100. Optionally, the catheter systems 10 described herein can include one or more other devices including one or more of a guidewire, access device (guide sheath), and interventional working devices.

The outer catheter 200 can be delivered using a tapered catheter 100 inserted through a lumen of the outer catheter 200. The tapered catheter 100 including the distal tip 110 and at least a length of the proximal section 105 up to the proximal luer 126 can be received within and extended through a lumen of another catheter, such as outer catheter 200 shown in FIGS. 2C-2D. The outer diameter of the cylindrical surface 107 of the proximal section 105 can be substantially size-matched to the outer catheter 200 providing a close fit that minimizes a distal lip or edge at the distal end 223 of the outer catheter 200. The close fit still allows for axial movement relative to one another so as to allow a user to achieve a desired extension or withdrawal of the tapered catheter 100 relative to the outer catheter 200 or the outer catheter 200 relative to the tapered catheter 100. The movement between the catheters that is allowed requires application of a relatively small load so as to avoid any negative impact on usability within a patient.

FIG. 2C illustrates a catheter system 10 having at least one inner catheter 100 (referred to herein also as a tapered catheter) and an outer catheter 200. The inner catheter 100 is receivable within a lumen (not shown) of the outer catheter 200 and can be advanced from the lumen of the outer catheter 200 into the narrowed portion of the blood vessel to provide the dilating function for treating the narrowed portion. The outer catheter 200 can be an aspiration catheter. The outer catheter 200 can be another type of catheter suitable for supporting advancement of one or more other working devices, such as a stent delivery system.

FIG. 2D illustrates a system 10 having two inner catheters 100a, 100b and an optional outer catheter 200 having a lumen sized to receive either of the two inner catheters 100a, 100b. The inner catheters 100a, 100b can be used to treat a narrowing in a blood vessel. The outer catheter 200 is optional and can be used to treat the same narrowing or a different narrowing. The treatment provided by the outer catheter 200 can involve aspiration or delivery of another tool to perform the treatment. The outer catheter 200 can be guided into position to perform its treatment by one of the inner catheters 100a, 100b. As an example, the inner catheters 100a, 100b can be used to treat a narrowed portion in a blood vessel that is due to atherosclerotic disease. The optional outer catheter 200 can be navigated to the narrowed portion treated by the inner catheters 100a, 100b or another narrowed portion in a different blood vessel, for example, distal to the first blood vessel. The outer catheter 200 can navigated into its treatment position by one of the inner catheters 100a, 100b. The two inner catheters 100a, 100b have different outer diameters along their proximal portions 105. The outer catheter 200 can be size-matched to the smaller inner catheter 100a, preferably the outer catheter 200 is size-matched to the larger inner catheter 100b.

As an example method of using the system 10 of FIG. 2D, the first inner catheter 100a can be inserted into a narrowed portion of a blood vessel, such as a carotid or cerebral artery. The first inner catheter 100a can be advanced to a dilating position and then removed from the narrowed portion. The second inner catheter 100b can thereafter be inserted into the narrowed portion of the blood vessel and advanced to a dilating position. The outer catheter 200 can be a full-length catheter for any of a variety of purposes. Alternatively, the outer catheter 200 can be a partial-length catheter, such as those described in U.S. Pat. No. 11,020,133, which is incorporated by reference herein in its entirety. The outer catheter 200 can be an aspiration catheter, stent delivery catheter, stent retriever delivery catheter, or another catheter. As an example, the first inner catheter 100a and/or the second inner catheter 100b can dilate the narrowed portion of the blood vessel according to a treatment protocol as a first-line and only treatment of the narrowing. The outer catheter 200 can deliver a second-line treatment, such as aspiration before, during, and/or after dilating the narrowed portion with the inner catheter 100. Still further, the outer catheter 200 can be used to deliver a second-line treatment, such as delivering a stent to the narrowed portion previously dilated by the inner catheter 100. The outer catheter 200 is capable of being navigated to the narrowed portion. The outer catheter 200 can be navigated over the second inner catheter 100b, or navigated together with the second inner catheter 100b. As will be described in more detail below, the distal tip 110 of the second inner catheter 100b can extend distal to the distal end 223 of the outer catheter 200 during navigation together. The outer catheter 200 is adapted to be advanced through the narrowed portion to distal sites, such as a cerebral occlusion distal to the narrowed portion to treat the cerebral occlusion.

The catheter systems 10 described herein can perform treatments that include dilation of the narrowed portion alone, dilation plus stenting of the narrowed portion, dilation of the narrowed portion plus distal occlusion treatment, dilation plus stenting of the narrowed portion, and/or distal occlusion treatment in the same procedure or in separate procedures. It should also be appreciated that the catheter systems 10 described herein can perform treatments using any variety of combination of sizes of catheters as discussed above.

In some implementations, the catheter system 10 can be a single operator system such that each of the components and systems can be delivered and used together by one operator through a single point of manipulation requiring minimal hand movements such that all wire and catheter manipulations can occur at or in close proximity to a single rotating hemostatic valve (RHV) or more than a single RHV co-located in the same device.

The flexibility and deliverability of the outer catheter 200 allows the outer catheter 200 to take the shape of the tortuous anatomy and avoids exerting straightening forces creating new anatomy. The outer catheter 200 is capable of this even in the presence of the inner catheter 100 extending through its lumen. Thus, the flexibility and deliverability of the inner catheter 100 is and the flexibility and deliverability of the outer catheter 200 are configured to reach the middle cerebral artery (MCA) circulation without straightening out the curves of the anatomy along the way.

The catheter system 10 can be a distal access system that extends to a target treatment site from a point of entry at the percutaneous arteriotomy (e.g. the femoral artery or other point of entry including radial artery, ulnar artery, brachial artery of an arm, and the like, or a corresponding vein). Conventional distal access systems for neurointervention typically include a long guide sheath or guide catheter placed through a shorter “introducer” sheath (e.g., 11-30 cm in length) at the groin. The long guide sheath is typically positioned in the ICA to support neurovascular interventions including stroke embolectomy (sometimes referred to as “thrombectomy”). For added support, these can be advanced up to the bony terminal petrous and, rarely, into the cavernous or clinoid or supraclinoid terminal ICA when possible. In some implementations, the guide sheath can be positioned lower within the carotid artery depending on whether the carotid is occlusive and being treated. To reach targets in the M1 or M2 distribution with devices for certain procedures an additional catheter may be inserted through the long guide catheter.

Still with respect to FIG. 2C, the outer catheter 200 can include a relatively flexible, luminal portion having a single, inner lumen (i.e., no more than one lumen) between a proximal opening 222 into the lumen and a distal opening 224 from the lumen located near or at the distal-most end 223 of the catheter 200. The outer catheter 200 can have one or more radiopaque markers 230, such as a radiopaque marker 230 located near the distal-most end 223 of the catheter 200 to aid in navigation and proper positioning of the distal-most end 223 under fluoroscopy. The radiopaque marker(s) 230 of the outer catheter 200, particularly those near the distal end region navigating extremely tortuous anatomy, can be relatively flexible such that they do not affect the overall flexibility of the catheter 200. The radiopaque marker(s) 230 can be tungsten-loaded or platinum-loaded markers that are relatively flexible compared to other types of radiopaque markers used in devices where flexibility is not paramount. In some implementations, the radiopaque marker 230 can be a band of tungsten-loaded PEBAX having a durometer of Shore 35D.

The outer catheter 200 can be used for a variety of interventional procedures, such as delivering endovascular scaffolding devices to the intracranial anatomy or for delivering aspiration. Where the outer catheter 200 is an aspiration catheter, such as for removing thrombotic material via aspiration, or for the prevention of embolic particles to flow downstream during placement of endovascular devices, such as stents across an occlusion due to atherosclerotic disease, it is desirable to that the catheter have as large a bore as clinically possible to achieve an optimal result for aspiration. Large-bore and super-bore catheters are particularly useful for these purposes. Catheter inner diameter can be maximized for treating these locations to obtain more beneficial fluid dynamics for aspirating and removing thrombotic material. For example, a distal access catheter can have an inner diameter of at least about 0.070″ or 0.088″ or greater. For delivery of endovascular devices, the delivery catheter can have a large-enough bore to deliver the desired devices. In an embodiment, the outer catheter 200 can be about 0.088″ inner diameter.

Whether the outer catheter 200 is used to deliver aspiration or to deliver a working device, such as a stent, the outer catheter 200 can be used with a size-matched inner catheter 100 to aid in its delivery to distal sites. The inner catheter 100 allows for safer and more efficient delivery of large-bore and or super-bore catheters 200 to distal sites of the cerebral arteries. At least a portion of the distal tip 110 of the inner catheter 100 can be designed to extend distal to the distal end 223 of the outer catheter 200 during use of the catheter system 10. The distal tip 110 can protrude distally from the distal end 223 of the catheter 200 during advancement of the catheter 200 through the tortuous anatomy of the cerebral vessels as will be described in more detail below.

The inner catheter 100 with the distal tip 110 has a benefit over, for example, a conventional microcatheter for advancing an outer catheter 200 in that the proximal section 105 can have a relatively large outer diameter that is just 0.003″-0.010″ (0.0762 mm-0.254 mm) smaller than the inner diameter of the outer catheter 200 it is used with and still maintain a high degree of flexibility for navigating tortuous anatomy. When the gap between the two components is too tight (e.g., less than about 0.003″/0.0762 mm), the force needed to slide the inner catheter 100 relative to the outer catheter 200 can result in damage to one or both of the components and increases risk to the patient during the procedure. The gap results in too tight of a fit to provide optimum relative sliding. When the gap between the two components is too loose (e.g., greater than about 0.010″/0.254 mm), the distal end 223 of the outer catheter 200 forms a lip that is prone to catch on carotid dissections or branching vessels during advancement through tortuous neurovasculature, such as around the carotid siphon where the ophthalmic artery branches off.

Depending upon the inner diameter of the outer catheter 200, the difference between the inner diameter of outer catheter 200 and the outer diameter of the cylindrical section 107 of the inner catheter 100 along at least a portion of its length, such as at least 10 cm of its length, preferably at least 15 cm of its length can be no more than about 0.015″ (0.381 mm), such as within a range of about 0.003″-0.015″ (0.0762 mm-0.381 mm) or between 0.006″-0.010″ (0.1524 mm-0.254 mm). Thus, the clearance between the inner and outer catheters 100, 200 can result in a space on opposite sides that is no more than about 0.008″ (0.2032 mm), or can be no more than about 0.005″ (0.127 mm), for example, from about 0.001″ up to about 0.006″ (0.0254 mm-0.1524 mm), preferably about 0.002″ to about 0.005″ (0.0508 mm-0.127 mm), and more preferably about 0.003″ to about 0.005″ (0.0762 mm-0.0508 mm).

The gap in ID/OD between the cylindrical surface 107 of the proximal section 105 and the outer catheter 200 can be in this size range (e.g., 0.003″-0.015″ (0.0762 mm-0.381 mm) or between 0.006″-0.010″ (0.152 mm-0.254 mm)) along a majority of their lengths. For example, the proximal section 105 of the inner catheter 100 can have a relatively uniform outer diameter that is between about 0.048″ (1.219 mm) to about 0.080″ (2.032 mm) from a proximal end region to a distal end region up to a point where the tapered portion 115 of the distal tip 110 begins. Similarly, the outer catheter 200 can have a lumen with a relatively uniform inner diameter that is between about 0.054″ (1.372 mm) to about 0.088″ (2.235 mm) from a proximal end region to a distal end region. As such, the difference between their respective inner and outer diameters along a majority of their lengths can be within this gap size range of 0.003″ to 0.015″ (0.0762 mm-0.381 mm). During use, the tapered portion 115 of the distal tip 110 is configured to extend distal to the distal end 223 of the outer catheter 200 such that the region of the inner catheter 100 having an outer diameter sized to match the inner diameter of the outer catheter 200 is positioned within the lumen of the outer catheter 200 such that the outer diameter of the inner catheter 100 can minimize the lip at the distal end 223 of the outer catheter 200.

The arrangement between the inner catheter 100 and the outer catheter 200 can be maintained during advancement of the catheter system 10 through the tortuous anatomy to reach the target location for treatment in the distal vessels and aids in preventing the distal end of the outer catheter 200 from catching on tortuous branching vessels. The inner catheter 100 can be in a juxtapositioned relative to the outer catheter 200 that provides an optimum relative extension between the two components for single smooth advancement. The inner catheter 100 can be positioned through the lumen of the outer catheter 200 such that its distal tip 110 extends just beyond a distal-most end 223 of the outer catheter 200. The distal tip 110 of the inner catheter 100 eliminates the stepped transition between the inner catheter 100 and the outer catheter 200 thereby avoiding issues with catching on branching vessels within the region of the vasculature such that the outer catheter 200 may easily traverse the multiple angulated turns of the carotid siphon. The optimum relative extension, for example, can be the distal tip 110 extending just distal to a distal-most end 223 of the outer catheter 200. A length of the distal tip 110 extending distal to the distal-most end 223 of the outer catheter 200 during advancement can be between 0.5 cm and about 4 cm.

The components can be advanced together (or sequentially) with a guidewire, over a guidewire pre-positioned, or without any guidewire at all. In some implementations, the guidewire can be pre-assembled with the inner catheter 100 and outer catheter 200 such that the guidewire 500 extends through the lumen 120 of the inner catheter 100, which is loaded through the lumen of the outer catheter 200, all prior to insertion into the patient. The pre-assembled components can be simultaneously inserted into the sheath and/or advanced together up through and past the turns of the carotid siphon. A guidewire may be located within the lumen 120 of the inner catheter 100.

Navigation using the inner catheter 100 can provide maximal deliverability with minimal vascular trauma of an outer catheter. Catheter systems can cause “razoring” effects in a curved vessel because the blunt end of a large bore outer catheter can tend to take the greater curve in rounding a vessel when pushed by the operator. This blunt end can gouge or “razor” the greater curve with its sharp edge increasing the risk for dissection along an anatomic plane within the multilayered mid- or large-sized artery or vein (see, e.g. Catheter Cardiovasc. Interv. 2014 February; 83(2):211-20). The inner catheter 100 can serve to minimize the edge of the outer catheter 200. Positioning the inner catheter 100 within the lumen 223 of the large-bore or super-bore outer catheter 200 such that the tapered portion 115 (e.g., identifiable by radiopaque marker 130) of the inner catheter 100 is aligned optimally with the distal end 223 of the outer catheter 200 (e.g., identifiable by radiopaque marker 230) minimizes the edge and thereby eliminates “razoring” as the large-bore or super-bore outer catheter 200 is advanced through turns of the vessel. This is particularly useful for the cerebral anatomy. For example, ICAD treatments are typically needed in regions distal to the carotid siphon, particularly distal to the ophthalmic artery takeoff from the greater curve of the severe tortuosity of the final turn of the carotid siphon “S-turn,” the “anterior genu” of the carotid siphon typically seen as part of the terminal internal carotid artery (ICA). The specifics of the inner catheter 100 in proper alignment within the large-bore outer catheter 200 (the “tip-to-taper” position noted by the distal tip marker) relative to the taper marker 130 of the inner catheter 100 maximize the likelihood that razoring and catching on the ophthalmic artery branch are avoided during manual advancement of the catheter system 10. In some methods, the taper marker 130 of the inner catheter 100 can be positioned at or past the take-off of the ophthalmic artery to minimize these deleterious effects and allow the outer catheter 200 to pass the ophthalmic artery without incident. In a relatively straight segment, which is common after passing the siphon, the outer catheter 200 can be advanced over the inner catheter 100, which serves still as a guiding element to the target. The transition between the inner catheter 100 and the distal edge 223 of the outer catheter 200 is insignificant, especially compared to step-changes that would present relative to a typical microcatheter or guidewire, which would not prevent hang-ups on branches such as the ophthalmic artery. The inner catheter 100 allows for maneuvering of the outer catheter 200, even large-bore or super-bore sized outer catheters, clear to the face of the occlusion without use of a microcatheter or guidewire and without crossing and/or fragmenting the occlusion to reach it.

The inner catheter 100 enables the outer catheter 200 to be delivered without a guidewire. This ability to deliver the outer catheter 200 without a guidewire (or with a guidewire located within the lumen 120 of the inner catheter 100 and parked proximal of the distal opening 124 for potential use) is based, in part, upon the smooth transitions between the outer diameter of the inner catheter 100 and the outer catheter 200 as well as the smooth transition in flexibility between the two. The inner catheter 100 is capable of bending into an arc of greater than 180 degrees, the softness and flexibility creates a smooth arc without severe bends or kinks in the geometry of the catheter. The distal tip 110 of the inner catheter 100 seeks the larger lumens of the vascular anatomy as opposed to into the smaller branch arteries and goes where the majority of blood flow goes. The distal tip 110 of the inner catheter 100 can facilitate a strong preference to seek out the larger vessels during advancement into the distal vessels. This propensity to stay within the main fluid channel allows for safer and quicker navigation of tortuous vessels as well as the advancement of large bore catheters without the aid of a guidewire. The propensity to follow the main channels of blood flow aligns with acute ischemic stroke pathophysiology where major emboli tend to follow these same routes to a point where the occlusion lodges and interrupts antegrade blood flow. As well, these major channels are often ideal for placement of access catheters as these conduit arteries allow for smaller catheters to pass into specific target arteries for therapeutic intervention.

Standard neurovascular intervention, and nearly all endovascular intervention, is predicated on the concept that a guidewire leads a catheter to a target location. The guidewires are typically pre-shaped and often find side-branches of off-target locations where the guidewire will bunch or prolapse causing time-consuming nuisances during interventions that often require repeated redirection of the guidewire by the operator to overcome. In addition, this propensity of a guidewire to enter side-branches can be dangerous. Guidewires are typically 0.014″ to 0.018″ (0.356 mm-0.457 mm) in the neuroanatomy and will find and often traumatize dissection flaps or small branches that accommodate this size, which can lead to small bleeds or dissections and further occlusion. In a sensitive area like the brain these events can be catastrophic. The tendency of a guidewire to bunch and prolapse can also cause a leading edge to the guidewire that can be advanced on its own or as part of a triaxial system to create dissection planes and traumatize small vessels. Guidewires are also designed to cross occlusions caused by an embolus or atherosclerotic disease, primarily for the purpose of securing the guidewire to provide support for delivery of a catheter over the guidewire. However, crossing the occlusion with the guidewire can increase a risk of dislodging embolic debris, which travels distal to the occlusion site creating additional occlusion sites. Guidewires also increase the risk of perforations.

In contrast, the inner catheter 100 described herein preferentially stays in the larger lumen of a conduit vessel. The inner catheter 100 delivers to the largest lumen within the anatomy even in light of the highly tortuous anatomy and curves being navigated. The inner catheter 100 can preferentially take the larger lumen at a bifurcation or dissection flap while also following the current of the greatest blood flow thereby maintaining the general direction and angulations of the parent vessel. In viewing the standard anatomy found in the cerebral vasculature, the Circle of Willis is fed by two vertebral and two carotid conduit arteries. As these four arteries are the access points to the cerebral anatomy—the course of the inner catheter 100 can be identified and has been validated in standard cerebral anatomy models.

In the anterior circulation where the conduit artery point of entry for cerebral endovascular procedures is the internal carotid artery (ICA), the inner catheter 100 can guide the large-bore or super-bore sized outer catheter 200 to the M1 segment of the middle cerebral artery (MCA) bypassing the anterior communicating artery (ACA) and anterior temporal branch (ATB). The very flexible nature of the inner catheter 100 combined with the distal flexible nature of most cerebral catheters combine to allow delivery through severe tortuosity. Independent of the tortuous nature of the course of the arteries, the inner catheter 100 tends to navigate the turns and deliver to the largest offspring from a parent artery, for example, ICA to M1 segment of the MCA. The M2 level branching of the M1 can be variable, but is often seen to have two major M2 branches (superior and inferior) and, depending on the anatomy, which can vary significantly between patients, may be seen to bifurcate “equally” or “unequally.” If the caliber of the M2 branching is of similar size and angulation, the inner catheter 100 may take one of the two branches. If the target for catheter placement is not in a favorable angulation or size of artery, the inner catheter 100 may be curved (e.g., via shaping of a malleable distal tip) and directed or a guidewire may be used.

In some anatomies where the M2 bifurcation is “even” in size, a back-and-forth motion may aid in selecting one branch then the other while still avoid the need or use of a guidewire or a curved distal tip of the inner catheter 100. The back-and-forth motion can allow for the inner catheter 100 to be directed into either branch of the M2. The inner catheter 100, even when initially straight, achieves some curvature that aids in directing it into a branch vessel. Thus, when an operator encounters an M2 bifurcation and there is a desire to cannulate either branch of an evenly divided bifurcation, selection of either branch is possible using the catheter 100 without a guidewire.

Thus, main channels such as the ICA and the middle cerebral artery and its tributaries in the anterior circulation will naturally be the pathway of preference for the described inner catheter 100 and subsequent large-bore or super-bore outer catheter 200 delivery (via access from the ICA). A similar phenomenon can occur in the posterior circulation, which is accessed via the vertebral arteries arising from the subclavian arteries on the right and the left. The inner catheter 100 will take the main channels in this circulation as well by traversing the vertebral arteries to the basilar artery and to the major tributaries of the basilar: the posterior cerebral artery and superior cerebellar arteries in the posterior circulation.

### Methods of Treatment

The catheters and catheter systems described herein can be used to access and treat various vascular pathologies, including blood vessels narrowed by atherosclerotic disease. The narrowed portion of the vessels are treated by dilation using a tapered catheter, alone or in conjunction with other treatments, such as sequential dilation of a narrowed portion using kits of catheters having ever larger maximum outer diameters, as well as treatments of distal occlusions including aspiration embolectomy and/or stent deployment. The catheters and catheter systems provide a safer way to navigate to target blood vessels, including partially occluded vessels, find a path through the partial occlusion, and widen that path. The devices, systems, and methods also enable safe and rapid positioning of large interventional devices, such as a large-bore and super-bore aspiration catheters or stent delivery catheters.

The treatments considered herein for treating blood vessels including arteries and veins of the head and neck region, such as the carotid and/or cerebral blood vessels, including a carotid artery, including an internal carotid artery, or a cerebral artery, including a middle cerebral artery, can include dilating the narrowed portion of the blood vessel with a catheter having a soft, atraumatic, flexible lubricious distal tip with a tapering design, such as any of the catheters illustrated in FIGS. 2A-2D, FIGS. 3A-3D, FIG. 4, or FIG. 5.

The blood vessel narrowing treated includes extracranial occlusions and intracranial occlusions (i.e., intracranial atherosclerotic disease (ICAD)), persistent flow-limiting stenosis after mechanical thrombectomy, restenotic lesions, and other types of blood vessel narrowing. ICAD can be defined by angiographic evidence of an occlusion, such as a tapered-shape occlusion, presence of arterial calcific atherosclerotic lesions, remnant stenosis greater than about 70% after initial aspiration thrombectomy. Other angiographic features, such as the presence of intracranial carotid artery atherosclerotic changes, presence of multifocal narrowing and/or the presence of leptomeningeal collateral.

### Methods of Dilation

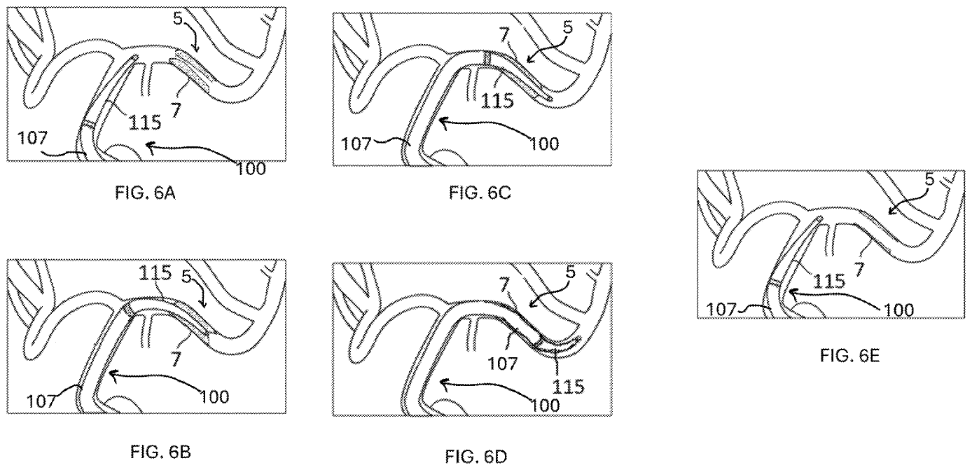

In an implementation, the method of treatment includes dilating the narrowed portion of the blood vessel with a tapered catheter 100 as shown, for example, in FIG. 2A or 2B and also in FIGS. 6A-6E. The narrowed portion 5 of the blood vessel is illustrated in FIGS. 6A-6E as being as a result of a lipid plaque 7 due to atherosclerosis. The tapered catheter 100 is navigated through a patient's vasculature to the narrowed portion 5 of a blood vessel (FIG. 6A). The catheter 100 can be advanced to the narrowed portion 5 through an access catheter positioned within an arteriotomy, or venotomy as described elsewhere herein. The distal tip 110 of the tapered catheter 100 is inserted into the narrowed portion 5. The tapered catheter 100 is advanced within the narrowed portion 5 so that the tapered portion 115 of the distal tip 110 passes at least partially through the narrowed portion 5 to dilate the narrowed portion 5 (FIG. 6B-6C). The tapered portion 115 can be advanced beyond the narrowed portion 5 such that an entirety of the distal tip 110 is advanced distal to the narrowed portion 5 so that the cylindrical surface 107 having the maximum dilating power is located within the narrowed portion 5. For example, a radiopaque marker identifying the tapered portion 115 (e.g., a proximal end of the tapered portion 115 or distal end region of the cylindrical surface 107 before the tapering) can be positioned just beyond a distal extension of the narrowed portion 5. The tapered catheter 100 is a non-expandable catheter having no more than a single lumen extending through it. The tapered catheter 100 provide the dilating function without any outward expansion, such an expandable balloon does, but rather due to the gradual increase in outer diameter along the tapered portion 115 of the distal tip 110 towards the larger outer diameter of the cylindrical surface 107. In a first optional alternative, the tapered catheter 100 can be advanced without a guidewire as described elsewhere herein. In a second optional alternative, the tapered catheter 100 can be advanced over a guidewire 500 that extends through the narrowed portion 5, the guidewire 500 being received within the lumen 120 of the tapered catheter 100.

As a further step in the treatment, the tapered catheter 100 can be maintained in a dilating position within the narrowed portion 5 for a dwell time (FIG. 6D). The dwell time can be selected so that at least a portion of the narrowed portion 5 is less narrow following removal of the tapered catheter 100 from the narrowed portion than the narrowed portion was prior to inserting the distal tip 110 of the tapered catheter 100 into the narrowed portion (FIG. 6E). The dilating position can be a position where the cylindrical surface 107 of the proximal section 105 of the tapered catheter 100 (i.e., the portion having the maximum dilating power due to its outer diameter being the largest of the catheter body and/or it having a hardness and/or stiffness greater than the tapered portion 115) is advanced into at least a portion of the narrowed portion 5.

The dwell time can vary, for example, due to the characteristics of the narrowing and/or the characteristics of the tapered catheter 100 selected for the dilation. In some implementations, the dwell time is greater than about 1 second, greater than about 2 seconds, greater than about 3 seconds, greater than about 4 seconds, greater than about 5 seconds, such as about 10 seconds-200 seconds, or about 60 seconds-120 seconds, and including any time in between. In other implementations, the dwell time is greater than about 3 seconds, greater than about 5 seconds, greater than about 10 seconds, greater than about 20 seconds, greater than about 30 seconds, such as about 10 seconds-200 seconds, about 30 seconds-150 seconds, or about 60 seconds-120 seconds, and including any time in between. In some implementations, the method is for treating an occlusion due to atherosclerotic disease and the dwell time is at least 20 seconds so that at least a portion of the narrowed portion is less narrowing following removal of the tapered catheter that the narrowed portion was prior to inserting the tapered catheter into the narrowed portion.

The boundaries of the narrowed portion 5, particularly on the downstream side, can be difficult to assess for an operator. At least a portion of the catheter 100 can be advanced to be within at least a portion of the narrowed portion 5 of the vessel and need not cross the entirety of the narrowed portion 5 to dilate it. For example, the tapered portion 115 can be advanced so that it passes beyond a narrowest part, whether or not that location is indeed distal to an entirety of the narrowed portion 5.

The catheter 100 can be advanced to and maintained in the dilating position at least once as shown in FIGS. 6A-6E. As a first optional step, the tapered portion 115 of the distal tip 110 of the catheter 100 can be removed from the narrowed portion as shown in FIG. 6E and readvanced within the narrowed portion. The catheter 100 readvanced to be within the narrowed portion can be maintained in the dilating position a second time. The dwell time of the second time the catheter is maintained in the dilating position can also vary including the same amount of time as the first dwell time or a different amount of time. The tapered portion 115 of the distal tip 110 can be removed from the narrowed portion of the blood vessel after the first dilation and the dilation of the narrowed portion assessed by injecting contrast medium as a second optional step. The catheter 100 can then be readvanced within the narrowed portion if the dilation is not a desired amount and the catheter maintained in the dilating position the second time. The tapered portion 115 of the distal tip 110 of the catheter can be removed from the narrowed portion of the blood vessel and repeatedly advanced into the narrowed portion and maintained in the dilating position until a desired dilation of the narrowed portion upon removal of the distal tip 110 from the narrowed portion is achieved.

The boundary of the narrowed portion 5 as well as the sufficiency of the dilation of the narrowed portion 5 can be assessed by angiography or other imaging techniques, including pre-procedural non-contrast computed tomography (CT), CT angiography, magnetic resonance angiography, digital subtraction angiography (DSA), positron emission tomography, and the like. Angiography involves injection of contrast media and use of x-ray fluoroscopic imaging to visualize internal anatomy of the vasculature to evaluate blood flow, constrictions, or blockage, and to plan an appropriate treatment. Contrast media is introduced prior to or during imaging (intra-arterially or intravenously). The presence of the contrast media blocks or limits the ability of the x-rays to pass through. As a result, any region that temporarily contains the contrast media changes its appearance on the images. The x-ray angiography provides high resolution imaging showing the vasculature anatomical details.

Contrast agent can be injected, such as through a guide sheath, into the vessel to visualize the narrowed portion 5 of the vessel and a baseline angiogram obtained. The baseline angiogram can be obtained, for example, in the anterior/posterior (AP) and/or lateral views. Fluoroscopic visualization may continue as the tapered catheter 100 is advanced and subsequent angiograms captured periodically, for example, before and after a dilation step to assess the size of the narrowed portion 5 and the sufficiency of the dilation achieved. The baseline angiogram image can be superimposed, such as with digital subtraction angiography, so that the vasculature and/or the narrowed portion are visible while the catheter(s) are present. Once the tapered catheter 100 is advanced into a dilating position and withdrawn according to a desired protocol, a confirmatory angiogram can be performed. The confirmatory angiogram can be performed by injecting contrast media to the narrowed portion 5, such as through the lumen of the tapered catheter 100 via the proximal luer, or through another catheter, such as the guide sheath used to access the narrowed portion 5.

The angiographic target for the treatment can vary according to current American Heart Association guidelines on the extended Thrombolysis in Cerebral Infarction (eTICI) scale. A successful reperfusion on the final angiogram following treatment can include a reperfusion grade of at least 2B (about 50-89% reperfusion), at least 2B50 (50%-66% reperfusion), at least 2B67 (67%-89% reperfusion), at least 2 C (about 90%-99% reperfusion), or 3 (about 100% reperfusion) on the eTICI scale.

In an interrelated implementation, the treatment includes dilating the narrowed portion with a tapered catheter 100 that is part of a system of similarly constructed catheters, such as shown the system 10 shown in FIG. 2B. The tapered catheter 100 can be part of a system of at least two tapered catheters in which a first tapered catheter 100a and a second tapered catheter 100b have different sized cylindrical surfaces 107. The system can include any of a variety of sizes used in combination with one another as described elsewhere herein.

The first tapered catheter 100a is navigated through a patient's vasculature to a narrowed portion 5 of a blood vessel similar to what is shown in FIG. 6A. The distal tip 110a of the first tapered catheter 100a is inserted into the narrowed portion 5 similar to what is shown in FIG. 6B. The first tapered catheter 100a is advanced within the narrowed portion 5 so that the tapered portion 115a of the distal tip 110a passes at least partially through the narrowed portion 5 to dilate the narrowed portion 5 similar to what is shown in FIG. 6C. The tapered portion 115a can be advanced beyond the narrowed portion 5 such that an entirety of the distal tip 110a is advanced distal to the narrowed portion 5 similar to what is shown in FIG. 6D. In a first optional alternative, the first tapered catheter 100a can be advanced without a guidewire as described elsewhere herein. In a second optional alternative, the first tapered catheter 100a can be advanced over a guidewire 500 that extends through the narrowed portion, the guidewire 500 being received within the lumen 120a of the first tapered catheter 100a.

The first tapered catheter 100a is removed from the narrowed portion similar to what is shown in FIG. 6E and the second tapered catheter 100b is navigated through the patient's vasculature to the narrowed portion of the blood vessel. The proximal section 105 of the second tapered catheter 100b has a cylindrical surface 107b having an outer diameter that is greater than the outer diameter of the cylindrical surface 107a of the first tapered catheter 100a and greater than an outer diameter of the tapered portion 115b of the distal tip 110b of the second tapered catheter 100b. The distal tip 110b of the second tapered catheter 100b is inserted into the narrowed portion 5 of the blood vessel that has been dilated by the first tapered catheter 100a. The second tapered catheter 100b can be advanced within the narrowed portion 5 so that the tapered portion 115b of the distal tip 110b of the second tapered catheter 100b passes at least partially through the narrowed portion 5 to dilate the narrowed portion 5. The second tapered catheter 100b is maintained in the dilating position within the narrowed portion 5 for a dwell time. The dwell time is selected as described elsewhere herein so that at least a portion of the narrowed portion 5 is less narrow following removal of the second tapered catheter 100b than it was prior to insertion of the second tapered catheter 100b into the narrowed portion 5.

The first tapered catheter 100a can be removed from the patient's vasculature before navigating the second tapered catheter 100b through the patient's vasculature.

The first tapered catheter 100a cylindrical surface 107a has an outer diameter that is smaller than the second tapered catheter 100b cylindrical surface 107b. As an example, the first tapered catheter 100a cylindrical surface 107a outer diameter can be about 10% to 40% smaller, or at least 20% smaller than the cylindrical surface 107b outer diameter of the second tapered catheter 100b. In an example, the first tapered catheter 100a cylindrical surface 107a outer diameter can be about 0.039 inches (1.0 mm)—about 0.071 inches (1.8 mm) and the second tapered catheter 100b cylindrical surface 107b outer diameter can be about 0.055 inches (1.4 mm)—about 0.088 inches (2.2 mm). In some implementations, the tapered portion 115 of the distal tip 110 can be from about 0.048″ outer diameter down to about 0.031″ (0.787 mm) outer diameter over a length of about 1 cm. In some implementations, the tapered portion 115 of the distal tip 110 can be from 0.062″ (1.575 mm) outer diameter to about 0.031″ (0.787 mm) outer diameter over a length of about 2 cm. In still further implementations, the tapered portion 115 of the distal tip 110 can be from 0.080″ (2.032 mm) outer diameter to about 0.031″ (0.787 mm) outer diameter over a length of about 2.5 cm. Any of a variety of sizes can be used in combination to perform the method as described herein.

The narrowed portion 5 treated by the tapered catheters 100 and catheter systems 10 described herein can be due to an occlusion that is a carotid artery stenosis or an occlusion in a cerebral blood vessel, including an occlusion due to atherosclerotic disease. The methods described herein can further include performing a treatment distal to the narrowed portion 5, such as treating an occlusion in a cerebral blood vessel that is distal to a narrowed portion 5 within a carotid artery. The methods described herein can further include navigating a second catheter 200 through the narrowed portion treated by at least one of the first and second tapered catheter(s) 100a, 100b to a site of a cerebral blood vessel occlusion and treating the occlusion with an outer catheter 200. The treatment of the occlusion with the outer catheter 200 can include treating the occlusion with aspiration where the outer catheter 200 is an aspiration catheter. Optionally, the outer catheter 200 is navigated to the cerebral blood vessel to perform the treatment of the occlusion over the first tapered catheter 100a or the second tapered catheter 100b, whichever catheter did the initial dilating of the narrowed portion. In preferred implementations, a first tapered catheter 100a is advanced to perform an initial dilation step and is removed. A larger, second tapered catheter 100b is advanced to perform a second dilating step and used to guide an outer catheter 200 over the larger, second tapered catheter 100b for advancing towards a distal occlusion. In an alternative implementation, a first tapered catheter 100a is advanced to perform an initial dilation step and is removed. A larger, second tapered catheter 100b is advanced to perform a second dilating step and is removed. The first tapered catheter 100a is reinserted and used to guide a smaller sized outer catheter 200. Any number of combinations of inner catheters 100 and outer catheter 200 are considered herein to perform dilation steps and advancement steps.

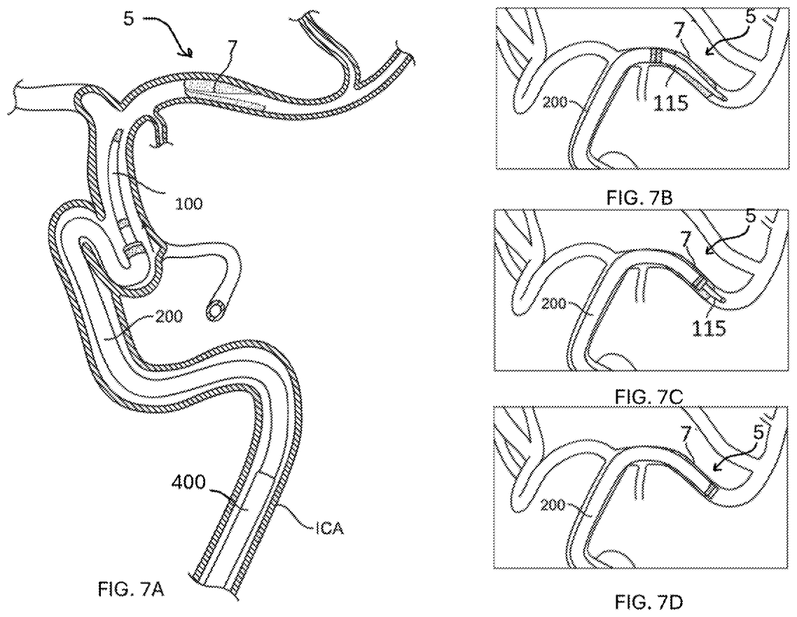

In another interrelated implementation, the treatment includes dilating the narrowed portion 5 with a tapered catheter 100 that is part of a system of catheters, such as shown the system 10 shown in FIGS. 2C-2D and also in FIGS. 7A-7D. The catheter 100 can be part of a system of at least two catheters having an inner catheter 100 that is receivable within a lumen 230 of an outer catheter 200. The system can include any of a variety of sizes used in combination with one another as described elsewhere herein.

The method includes navigating the outer catheter 200 to the narrowed portion 5 of the blood vessel as shown in FIG. 7A. The narrowed portion 5 of the blood vessel is illustrated in FIGS. 7A-7D as being as a result of a lipid plaque 7 due to atherosclerosis. The inner catheter 100 is advanced from the lumen 230 of the outer catheter 200 into the narrowed portion. The outer catheter 200 and the inner catheter 100 can be navigated to the narrowed portion together as shown in FIG. 7B. The inner catheter 100 can be navigated to the narrowed portion and the outer catheter 200 then navigated to the narrowed portion over the inner catheter 100 as shown in FIG. 7C. Optionally, where the inner and outer catheter 100, 200 are navigated together, the distal tip 110 of the inner catheter 100 can extend distal to the distal end 223 of the outer catheter 200 and the proximal segment 105 of the inner catheter 100 is positioned within the lumen of the outer catheter 200 during navigation.

The method can optionally include treatment of an occlusion, or other cerebrovascular disease including ischemic stroke, hemorrhagic stroke, cerebral aneurysm, vascular malformation, in a cerebral blood vessel distal to the narrowed portion. The outer catheter 200 can be navigated through the narrowed portion 5 to a site of a cerebral blood vessel occlusion and the occlusion treated. The treatment can include aspiration of the occlusion with an aspiration catheter. The aspiration catheter can be navigated over the inner catheter 100 that did the dilating of the narrowing as shown in FIG. 7C. The treatment can include optionally deploying a stent in the narrowed portion before or after treating the cerebral blood vessel occlusion. The treatment can optionally include deploying a stent in the narrowed portion after treating the cerebral blood vessel occlusion during the same surgical procedure. Optionally, the stent can be deployed in the narrowed portion after treating the cerebral blood vessel occlusion and in a different surgical procedure.

In the methods in which an outer catheter is used, the outer catheter 200 can be a conventional full-length catheter or a catheter having a larger diameter distal luminal portion coupled to a smaller diameter proximal control element as described in U.S. Pat. No. 11,020,133, or U.S. Publication No. 2023/0355255, filed May 3, 2023, each of which are incorporated by reference in their entireties herein, so that monopoint manipulation at the base sheath hub is possible.

In any of the methods described herein, a base sheath 400 (e.g., 6F, 7F, 8F, etc.) can be introduced into a blood vessel through an arteriotomy (e.g., femoral artery, or other arterial access site or venous access site where the blood vessel narrowing is within a vein) and advanced to the level of at least the common carotid artery, for example, to a location proximal of the bifurcation of the internal and external carotid arteries within the common carotid artery. The base sheath 400 can be any of a variety of sizes depending on the method to be performed. For example, if a large aspiration catheter is to be inserted, a larger sized base sheath 400 may be used. In implementations, where only a dilating tapered catheter is to be inserted to widen a narrowed portion of a vessel, a smaller sized base sheath 400 may be sufficient. The distal end of the base sheath 400 can also be advanced distal to the bifurcation into, for example, into a region of the internal carotid artery such as the cervical ICA as shown in FIG. 7A. A catheter, such as catheter 100 alone or a catheter 100 with an outer catheter 200, is advanced through the hub (e.g., an RHV) on the base sheath 400 until the distal end of the catheter 100 exits the distal opening of the base sheath 400.

The tapered catheter 100 can be used to treat the narrowed portion, such as by dilating the narrowed portion as described herein, without any further treatment of the narrowed portion. Alternatively, the tapered catheter 100 can be used to pre-dilate the narrowed portion prior to deploying a stent at the narrowed portion. Deploying the stent can be performed according to any of a variety of techniques. In an implementation, the outer catheter 200 can be delivered to the narrowed portion using the tapered catheter 100, which may be the same catheter that performed the pre-dilation, and a stent delivery system deployed through the outer catheter 200. For example, the outer catheter 200 can be positioned across the dilated narrowed portion and used to deploy, such as by unsleeving, an endovascular scaffolding device, such as a stent. Stent delivery is described in more detail in U.S. Publication No. 2023/0355255, filed May 3, 2023.

The catheters and catheter systems described herein can be advanced over a guidewire, such as a guidewire pre-positioned across an occlusion. Alternatively, the guidewire can be positioned within the catheter lumen 120 during advancement of the tapered catheter 100 such that the guidewire remains fully enclosed within the tapered catheter 100 (i.e., proximal to the distal opening 124 from the single lumen 120 of the tapered catheter 100).

In some patients, atherosclerotic disease is located within the extracranial anatomy leading to the more distal target sites within the intracranial anatomy. For example, at least a region of a carotid artery can be at least partially occluded. The extracranial occlusion can be treated prior to treating the intracranial occlusion. In some implementations, the extracranial occlusion can be treated by dilating the occlusion as the tapered catheter 100 navigates through the extracranial occlusion. The dilation of the occlusion in the extracranial anatomy can be sufficient to treat the occlusion such that no further treatments of the extracranial occlusion are needed. Alternatively, the occlusion in the extracranial anatomy can be treated using aspiration and/or delivery of a stent in addition to dilating the occlusion with the catheter. For example, the tapered catheter 100 can dilate the extracranial occlusion. The tapered catheter 100 that performed the dilation (or another larger catheter 100 exchanged for an initial dilating catheter 100) can be used to advance a large-bore or super-bore outer catheter 200 to the extracranial occlusion and/or to an intracranial occlusion distal to the extracranial occlusion. The tapered catheter 100 can be removed from the lumen of the large-bore or super-bore catheter and aspiration drawn through the lumen to aspirate the occlusion (and/or a distal occlusion). Alternatively, the tapered catheter 100 can be removed from the lumen of the large-bore or super-bore outer catheter and a stent delivery system advanced through the lumen to deploy a stent to the occlusion (and/or a distal occlusion).

### Materials

One or more components of the catheters described herein may include or be made from a variety of materials including one or more of a metal, metal alloy, polymer, a metal-polymer composite, ceramics, hydrophilic polymers, polyacrylamide, polyethers, polyamides, polyethylenes, polyurethanes, copolymers thereof, polyvinyl chloride (PVC), PEO, PEO-impregnated polyurethanes such as Hydrothane, Tecophilic polyurethane, Tecothane, PEO soft segmented polyurethane blended with Tecoflex, thermoplastic starch, PVP, and combinations thereof, and the like, or other suitable materials.

Some examples of suitable metals and metal alloys for catheter braid or coil reinforcement include stainless steel, such as 304V, 304L, and 316LV stainless steel; mild steel; nickel-titanium alloy such as linear-elastic and/or super-elastic Nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof, and the like; or any other suitable material and as described elsewhere herein.

Inner liner materials of the catheters described herein can include low friction polymers such as PTFE (polytetrafluoroethylene) or FEP (fluorinated ethylene propylene), PTFE with polyurethane layer (Tecoflex). Reinforcement layer materials of the catheters described herein can be incorporated to provide mechanical integrity for applying torque and/or to prevent flattening or kinking such as metals including stainless steel, Nitinol, Nitinol braid, helical ribbon, helical wire, cut stainless steel, or the like, or stiff polymers such as PEEK. Reinforcement fiber materials of the catheters described herein can include various high tenacity polymers like Kevlar, polyester, meta-para-aramide, PEEK, single fiber, multi-fiber bundles, high tensile strength polymers, metals, or alloys, and the like. Outer jacket materials of the catheters described herein can provide mechanical integrity and can be contracted of a variety of materials such as polyethylene, polyurethane, PEBAX, nylon, Tecothane, and the like. Other coating materials of the catheters described herein include paralene, Teflon, silicone, polyimide-polytetrafluoroetheylene, and the like. The inner liner may further include different surface finishes, such as dimples, bumps, ridges, troughs. The surface finishes may be randomly disposed, linearly disposed, spirally disposed, or otherwise disposed using a specific pattern along the length of the catheter. It is further contemplated that the inner liner may include a mixture of different surface finishes, for example, one section may have dimples, another section may have troughs, etc. Additionally, the surface finish may be incorporated along the entire length of the catheter or only in sections of the catheter. It is also contemplated that the inner liner may further include an electrosprayed layer, whereby materials could be incorporated into the inner liner. Examples of materials can include low friction materials as described above. Alternatively, the electrosprayed or electrospun layer may incorporate a beneficial agent that becomes free from the coating when exposed to blood, or to compression from a clot, for example, the beneficial agent may be a tissue plasminogen activator (tPA) or heparin encased in alginate.

Implementations describe catheters and delivery systems and methods to deliver catheters to target anatomies. However, while some implementations are described with specific regard to delivering catheters to a target vessel of a neurovascular anatomy such as a cerebral vessel, the implementations are not so limited and certain implementations may also be applicable to other uses. For example, the catheters can be adapted for delivery to different neuroanatomies, such as subclavian, vertebral, carotid vessels as well as to the coronary anatomy or peripheral vascular anatomy, to name only a few possible applications. It should also be appreciated that although the systems described herein are described as being useful for treating a particular condition or pathology, that the condition or pathology being treated may vary and are not intended to be limiting.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the catheters and/or delivery systems to a specific configuration described in the various implementations.

The dimensions provided herein are approximate and each dimension may have an engineering tolerance or a permissible limit of variation. The word “about” or “approximately” or “substantially” are intended to provide such permissible tolerance to the dimension being referred to and means a range of values including the specified value, which a person of ordinary skill in the art would consider reasonably similar to the specified value. In embodiments, about means within a standard deviation using measurements generally acceptable in the art. Where “about” or “approximately” or “substantially” is not used with a particular dimension herein that that dimension need not be exact. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The catheter system disclosed herein may be packaged together in a single package, where the catheters are packaged in a coil tube. The finished package would be sterilized using sterilization methods such as Ethylene oxide or radiation and labeled and boxed. Instructions for use may also be provided in-box or through an internet link printed on the label.

What is claimed is:

1. A method of treating a blood vessel that has been narrowed by atherosclerotic disease, wherein the blood vessel is a carotid blood vessel or a cerebral blood vessel, the method comprising:
navigating a catheter through a patient's vasculature to a narrowed portion of the blood vessel, the catheter having a proximal section and a distal tip having a tapered portion, wherein the proximal section has a cylindrical surface having an outer diameter greater than an outer diameter of the tapered portion of the distal tip, wherein the proximal section is capable of advancing the catheter through the patient's vasculature, and wherein the distal tip has a size and a flexibility to reach the narrowed portion of the blood vessel;
inserting the distal tip of the catheter into the narrowed portion of the blood vessel;
advancing the catheter within the narrowed portion so that the tapered portion of the distal tip passes at least partially through the narrowed portion to dilate the narrowed portion; and
maintaining the catheter in a dilating position within the narrowed portion for a dwell time selected so that at least a portion of the narrowed portion is less narrow following removal of the catheter than the narrowed portion was prior to inserting the distal tip of the catheter into the narrowed portion.

2. The method of claim 1, wherein the distal tip is non-expandable.

3. The method of claim 1, wherein the dilating position is a position where the cylindrical surface of the proximal section is advanced into at least a portion of the narrowed portion.

4. The method of claim 3, wherein the cylindrical surface is non-expandable.

5. The method of claim 1, wherein the dilating position is a position where the tapered portion is advanced entirely through the narrowed portion and the cylindrical surface of the proximal section is advanced into the narrowed portion.

6. The method of claim 1, wherein the outer diameter of the cylindrical surface is selected in relation to a diameter of the blood vessel when not narrowed and/or in relation to a characteristic of the atherosclerotic disease.

7. The method of claim 1, wherein the dwell time is greater than about 1 second.

8. The method of claim 1, wherein the dwell time is from about 10 seconds to about 200 seconds.

9. The method of claim 1, wherein the dwell time is from about 60 seconds to about 120 seconds.

10. The method of claim 1, comprising removing the tapered portion of the distal tip from at least a portion of the narrowed portion of the blood vessel, readvancing the catheter within the narrowed portion, and maintaining the catheter in the dilating position a second time.

11. The method of claim 1, comprising removing the tapered portion of the distal tip from the narrowed portion of the blood vessel, assessing the dilation of the narrowed portion, and readvancing the catheter within the narrowed portion if the dilation is not a desired amount, and maintaining the catheter in the dilating position a second time.

12. The method of claim 1, comprising removing the tapered portion of the distal tip from the narrowed portion of the blood vessel, assessing the dilation of the narrowed portion by injecting contrast medium, and readvancing the catheter within the narrowed portion if the dilation is not a desired amount, and maintaining the catheter in the dilating position a second time.

13. The method of claim 1, comprising removing the tapered portion of the distal tip from at least a portion of the narrowed portion of the blood vessel, and repeatedly advancing the catheter within the narrowed portion and maintaining the catheter in the dilating position until a desired dilation of the narrowed portion of the blood vessel upon removal of the distal tip from the narrowed portion is achieved.

14. The method of claim 1, wherein advancing the catheter within the narrowed portion comprises advancing the catheter without a guidewire.

15. The method of claim 1, wherein advancing the catheter within the narrowed portion comprises advancing the catheter over a guidewire that extends through the narrowed portion, the guidewire being received within a lumen of the catheter.

16. The method of claim 15, wherein the lumen has diameter less than about 0.024″.

17. The method of claim 15, wherein the lumen has a diameter of from about 0.014″ to about 0.024″.

18. The method of claim 1, wherein the catheter is a first catheter and wherein the method further comprises:
removing the first catheter from the narrowed portion;
navigating a second catheter through the patient's vasculature to the narrowed portion of the blood vessel, the second catheter having a proximal section and a distal tip having a tapered portion, wherein the proximal section of the second catheter has a cylindrical surface having an outer diameter greater than the outer diameter of the cylindrical surface of the first catheter and greater than an outer diameter of the tapered portion of the distal tip of the second catheter, wherein the proximal section is capable of advancing the second catheter through the patient's vasculature, and wherein the distal tip of the second catheter has a size and a flexibility to reach the narrowed portion of the blood vessel;
inserting the distal tip of the second catheter into the narrowed portion of the blood vessel that has been dilated by the first catheter;
advancing the second catheter within the narrowed portion so that the tapered portion of the distal tip of the second catheter passes at least partially through the narrowed portion to dilate the narrowed portion; and
maintaining the second catheter in a dilating position within the narrowed portion for a dwell time selected so that at least a portion of the narrowed portion is less narrow following removal of the second catheter than it was prior to insertion of the second catheter into the narrowed portion.

19. The method of claim 18, wherein the first catheter is removed from the patient's vasculature before navigating a second catheter through the patient's vasculature.

20. The method of claim 18, wherein the outer diameter of the cylindrical surface of the first catheter is from about 0.039 inches (1.0 mm) to about 0.071 inches (1.8 mm), and wherein the outer diameter of the cylindrical surface of the second catheter is from about 0.055 inches (1.4 mm) to about 0.088 inches (2.2 mm).

21. The method of claim 1, wherein the proximal section comprises a tubular portion and a proximal extension extending proximally from the tubular portion, wherein the proximal extension is less flexible than the tubular portion.

22. The method of claim 1, wherein the distal tip is from about 1 cm to about 5 cm long.

23. The method of claim 1, wherein the tapered portion of the distal tip is from about 0.5 cm to about 4 cm long.

24. The method of claim 1, wherein the distal tip comprises a first radiopaque marker positioned to indicate a distal end of the distal tip and a second radiopaque marker positioned to indicate a proximal end of the tapered portion.

25. The method of claim 1, wherein the distal tip comprises a cylindrical extension extending distal to the tapered portion.

26. The method of claim 1, wherein the distal tip is formed of a material having a material hardness of no greater than about Shore 35D.

27. The method of claim 1, wherein the tapered portion of the distal tip tapers from a first outer diameter to a second outer diameter that is smaller than the first outer diameter, wherein the first outer diameter is at least about 1.5 times the second outer diameter.

28. The method of claim 1, wherein the tapered portion of the distal tip tapers from a first outer diameter to a second outer diameter that is smaller than the first outer diameter, wherein the first outer diameter is from about 0.048 inches (1.22 mm) to about 0.080 inches (2.03 mm).

29. The method of claim 1, wherein the catheter is an inner catheter that is receivable within a lumen of an outer catheter, wherein the outer catheter is navigated to the narrowed portion of the blood vessel, and wherein the inner catheter is advanced from the lumen of the outer catheter into the narrowed portion of the blood vessel.

30. The method of claim 29, wherein the outer catheter and the inner catheter are navigated to the narrowed portion of the blood vessel together.

31. The method of claim 30, wherein the outer catheter has a distal end and wherein the distal tip of the inner catheter extends distal to the distal end of the outer catheter and the proximal section of the inner catheter is within the lumen of the outer catheter during navigation.

32. The method of claim 29, wherein the inner catheter is navigated to the narrowed portion of the blood vessel, and the outer catheter is then navigated to the narrowed portion over the inner catheter.

33. The method of claim 1, wherein the narrowed portion is a carotid artery stenosis.

34. The method of claim 1, further comprising treating an occlusion in a cerebral blood vessel distal to the narrowed portion.

35. The method of claim 1, further comprising navigating the catheter through the narrowed portion to a site of a cerebral blood vessel occlusion and treating the cerebral blood vessel occlusion.

36. The method of claim 35, wherein treating the cerebral blood vessel occlusion comprises treating with an aspiration catheter navigated to the cerebral blood vessel occlusion over the catheter.

37. The method of claim 36, further comprising deploying a stent in the narrowed portion before or after treating the cerebral blood vessel occlusion.

38. The method of claim 36, further comprising deploying a stent in the narrowed portion after treating the cerebral blood vessel occlusion during the same surgical procedure.

39. The method of claim 1, further comprising deploying a stent in the narrowed portion.

40. The method of claim 1, wherein the blood vessel is a carotid artery.

41. The method of claim 40, wherein the carotid artery is a common carotid artery, an internal carotid artery, or an external carotid artery.

42. The method of claim 1, wherein the blood vessel is a cerebral artery.

43. The method of claim 42, wherein the cerebral artery is a middle cerebral artery, an anterior cerebral artery, or a posterior cerebral artery.

44. A system for treating a carotid or cerebral blood vessel that has been narrowed, the system comprising:
a first catheter having a proximal section and a distal tip having a tapered portion, wherein the proximal section has a cylindrical surface having an outer diameter greater than an outer diameter of the tapered portion of the distal tip, wherein the proximal section is capable of advancing the first catheter through a patient's vasculature to a narrowed portion of the carotid or cerebral blood vessel, and wherein the distal tip has a size and a flexibility to reach the narrowed portion;
a second catheter having a proximal section and a distal tip having a tapered portion, wherein the proximal section has a cylindrical surface having an outer diameter greater than an outer diameter of the tapered portion of the distal tip, wherein the proximal section is capable of advancing the second catheter through the patient's vasculature to the narrowed portion of the carotid or cerebral blood vessel, and wherein the distal tip has a size and a flexibility to reach the narrowed portion; and
an outer catheter having a lumen sized to receive the second catheter, wherein the outer catheter is capable of being navigated to the narrowed portion of the blood vessel over the second catheter or together with the second catheter,
wherein the outer diameter of the cylindrical surface of the second catheter is greater than the outer diameter of the cylindrical surface of the first catheter so that the distal tip of the first catheter is insertable into the narrowed portion of the carotid or cerebral blood vessel, advanced to a dilating position, and removed from the narrowed portion and thereafter the second catheter is insertable into the narrowed portion of the carotid or cerebral blood vessel and advanced to a dilating position.

45. The system of claim 44, wherein the outer catheter is adapted to be advanced through the narrowed portion dilated by at least one of the first catheter and the second catheter to a cerebral occlusion distal to the narrowed portion to treat the cerebral occlusion.

46. The system of claim 45, wherein the outer catheter is an aspiration catheter.

47. The system of claim 44, wherein the outer catheter is adapted to deliver a stent to the narrowed portion.

48. The system of claim 44, wherein the outer catheter is adapted to be advanced through the narrowed portion to a cerebral occlusion distal to the narrowed portion to treat the cerebral occlusion and to deliver a stent to the narrowed portion.

49. The system of claim 44, wherein the outer catheter and the second catheter are adapted to be navigated together with the distal tip of the second catheter extending distal to a distal end of the outer catheter.

50. The system of claim 44, wherein the distal tip of the first catheter is non-expandable.

51. The system of claim 44, wherein the distal tip of the second catheter is non-expandable.

52. The system of claim 44, wherein the cylindrical surface of the first catheter is non-expandable.

53. The system of claim 44, wherein the cylindrical surface of the second catheter is non-expandable.

54. The system of claim 44, wherein the outer diameter of the cylindrical surface of the first catheter is from about 0.039 inches (1.0 mm) to about 0.071 inches (1.8 mm), and wherein the outer diameter of the cylindrical surface of the second catheter is from about 0.055 inches (1.4 mm) to about 0.088 inches (2.2 mm).

55. The system of claim 44, wherein the proximal section of the first catheter comprises a tubular portion and a proximal extension extending proximally from the tubular portion, wherein the proximal extension is less flexible than the tubular portion.

56. The system of claim 55, wherein the proximal section of the second catheter comprises a tubular portion and a proximal extension extending proximally from the tubular portion of the second catheter, wherein the proximal extension of the second catheter is less flexible than the tubular portion of the second catheter.

57. The system of claim 44, wherein the distal tip of the first catheter is from about 1 cm to about 5 cm long.

58. The system of claim 44, wherein the tapered portion of the distal tip of the first catheter is from about 0.5 cm to about 4 cm long.

59. The system of claim 44, wherein the distal tip of the first catheter comprises a first radiopaque marker positioned to indicate a distal end of the distal tip of the first catheter and a second radiopaque marker positioned to indicate a proximal end of the tapered portion of the first catheter.

60. The system of claim 44, wherein the distal tip of the first catheter comprises a cylindrical extension extending distal to the tapered portion of the first catheter.

61. The system of claim 44, wherein the distal tip of the first catheter is formed of a material having a material hardness of no greater than about Shore 35D.

62. The system of claim 44, wherein the tapered portion of the distal tip of the first catheter tapers from a first outer diameter to a second outer diameter that is smaller than the first outer diameter, wherein the first outer diameter is at least about 1.5 times the second outer diameter.

63. The system of claim 44, wherein the tapered portion of the distal tip of the first catheter tapers from a first outer diameter to a second outer diameter that is smaller than the first outer diameter, wherein the first outer diameter is from about 0.048 inches (1.22 mm) to about 0.080 inches (2.03 mm).

64. The system of claim 44, wherein the proximal section of the first catheter and the proximal section of the second catheter each comprise a tubular portion and a proximal extension extending proximally from the tubular portion, wherein the proximal extension is less flexible than the tubular portion.

65. The system of claim 44, wherein the distal tip of the first catheter and the distal tip of the second catheter are from about 1 cm to about 5 cm long.

66. The system of claim 44, wherein the tapered portion of the distal tip of the first catheter and the distal tip of the second catheter are from about 0.5 cm to about 4 cm long.

67. The system of claim 44, wherein the distal tip of the first catheter comprises a first radiopaque marker positioned to indicate a distal end of the distal tip of the first catheter and a second radiopaque marker positioned to indicate a proximal end of the tapered portion of the first catheter, and wherein the distal tip of the second catheter comprises a first marker band positioned to indicate a distal end of the distal tip of the second catheter and a second marker band positioned to indicate a proximal end of the tapered portion of the second catheter.

68. The system of claim 44, wherein the distal tip of the first catheter and/or the distal tip of the second catheter comprise a cylindrical extension extending distal to the tapered portion of the first catheter.

69. The system of claim 44, wherein the distal tip of the first catheter and the distal tip of the second catheter is formed of a material having a material hardness of no greater than about Shore 35D.

70. The system of claim 44, wherein the tapered portion of the distal tip of the first catheter and the tapered portion of the distal tip of the second catheter taper from a first outer diameter to a second outer diameter that is smaller than the first outer diameter, wherein the first outer diameter is at least about 1.5 times the second outer diameter.

71. The system of claim 44, wherein the first catheter and the second catheter are packaged together in a kit.

72. A sterile packaged kit of neurovascular tapered catheters, the kit comprising:
a first tapered catheter having a proximal section and a distal tip having a tapered portion, wherein the proximal section is capable of advancing the first tapered catheter through a patient's vasculature to a narrowed portion of a blood vessel, and wherein the distal tip has a size and a flexibility to reach the narrowed portion;
a second tapered catheter having a proximal section and a distal tip having a tapered portion, wherein the proximal section is capable of advancing the second tapered catheter through the patient's vasculature to the narrowed portion of the blood vessel, and wherein the distal tip has a size and a flexibility to reach the narrowed portion; and
wherein a maximum outer diameter of the second tapered catheter is greater than a maximum outer diameter of the first tapered catheter so that the distal tip of the first catheter is insertable into the narrowed portion of the blood vessel, advanced to a dilating position, and removed from the narrowed portion, and thereafter the second catheter is insertable into the narrowed portion of the blood vessel and advanced to a dilating position.

73. A method for treating a blood vessel that has been narrowed due to atherosclerosis, the method comprising:
navigating a catheter through a patient's vasculature to a narrowed portion of the blood vessel, the catheter having a proximal section and a distal tip having a tapered portion, wherein the proximal section has a cylindrical surface having an outer diameter greater than an outer diameter of the tapered portion of the distal tip, wherein the proximal section is capable of advancing the catheter through the patient's vasculature, and wherein the distal tip has a size and a flexibility to reach the narrowed portion of the blood vessel;
inserting the distal tip of the catheter into the narrowed portion of the blood vessel;
advancing the catheter to a dilating position that is within the narrowed portion so that the tapered portion of the distal tip passes at least partially through the narrowed portion to dilate the narrowed portion; and
maintaining the catheter in the dilating position within the narrowed portion for a dwell time of at least 20 seconds so that at least a portion of the narrowed portion is less narrow following removal of the catheter than the narrowed portion was prior to inserting the distal tip of the catheter into the narrowed portion.

74. The method of claim 73, wherein the blood vessel is of a head or neck of a patient.

75. The method of claim 73, wherein the blood vessel is a peripheral blood vessel or a coronary blood vessel.

76. The method of claim 75, wherein the peripheral blood vessel is one in an extremity, including a hand or a foot.

77. A method of treating atherosclerotic disease, the method comprising:
navigating a catheter through a patient's vasculature to a first narrowed portion in a carotid artery, wherein the first narrowed portion in the carotid artery is an occlusion due to carotid artery disease, wherein the catheter has a proximal section and a distal tip having a tapered portion, wherein the proximal section has a cylindrical surface having an outer diameter greater than an outer diameter of the tapered portion of the distal tip, wherein the proximal section is capable of advancing the catheter through the patient's vasculature, and wherein the distal tip has a size and a flexibility to reach the first narrowed portion;
inserting the distal tip of the catheter into the first narrowed portion;
advancing the catheter within the first narrowed portion so that the tapered portion of the distal tip passes at least partially through the first narrowed portion to dilate the first narrowed portion;
maintaining the catheter in a dilating position within the first narrowed portion for a dwell time selected so that at least a portion of the first narrowed portion is less narrow following removal of the catheter than the first narrowed portion was prior to inserting the distal tip of the catheter into the first narrowed portion; and
advancing the catheter towards a second narrowed portion, wherein the second narrowed portion is in an intracranial vessel and the second narrowed portion is due to intracranial atherosclerotic disease.
